(19) United States
(12) Patent Application Publication
(10) Pub. No.: US 2023/0201572 A1
(43) Pub. Date: Jun. 29, 2023

(54) CONDUCTIVE SPACER IN AN ELECTRODE ASSEMBLY OF AN ELECTRICAL TREATMENT APPARATUS

(71) Applicant: Pulse Biosciences, Inc., Hayward, CA (US)

(72) Inventors: Cameron D. Hinman, Thurman, NC (US); Kenneth R. Krieg, Fremont, CA (US)

(73) Assignee: Pulse Biosciences, Inc., Hayward, CA (US)

(21) Appl. No.: 18/088,157

(22) Filed: Dec. 23, 2022

Related U.S. Application Data
(63) Continuation of application No. PCT/US2022/024283, filed on Apr. 11, 2022.
(60) Provisional application No. 63/174,210, filed on Apr. 13, 2021.

(51) Int. Cl. A61N 1/04; A61N 1/05

(52) U.S. Cl. CPC A61N 1/0496; A61N 1/0476; A61N 1/0502

(57) ABSTRACT

To distribute electrical treatment to a treatment area of a patient, described herein are electrical therapy devices, methods of their operation and methods for delivery of the electrical therapy to the patient. In some embodiments, the electrical therapy device comprises an electrode assembly that includes at least two electrodes, and a conductive spacer positioned between the electrodes. Methods of selection of the electrical therapy devices and methods of their operation are also provided.

### CROSS REFERENCE TO RELATED APPLICATIONS

This application is a bypass continuation of and claims priority to PCT Application No. PCT/US2022/024283, filed Apr. 11, 2022, entitled “CONDUCTIVE SPACER IN AN ELECTRODE ASSEMBLY OF AN ELECTRICAL TREATMENT APPARATUS,” which claims priority to U.S. Provisional Application No. 63/174,210, filed Apr. 13, 2021, entitled “CONDUCTIVE SPACER IN AN ELECTRODE ASSEMBLY OF AN ELECTRICAL TREATMENT APPARATUS,” each of which are incorporated herein by reference in their entirety for all purposes.

### BACKGROUND

### Technical Field

Described herein are electrode assemblies (e.g., electrical applicator tips) that may be preferentially used to apply high voltage, ultra-short electrical pulses, such as nanosecond pulses, to treat patients. More specifically, described herein are electrode assemblies having conductive spacers and methods of using them for electrical therapy to distribute the electrical treatment more uniformly to the treatment area.

### Background

Electrical therapy treatments, particularly ultra-short, high-field strength electric pulses, also sometimes described as nanosecond Pulsed Electric Field (nsPEF) treatments, may be used for electromanipulation of biological cells. For example, electric pulses may be used in treatment of human or animal cells and tissue including tumor cells, such as basal cell carcinoma, squamous cell carcinoma, and melanoma.

These electrical therapy treatments are often performed using electrode assemblies (e.g., treatment tips) that either incorporate surface electrodes, which are placed on the treatment area without penetrating the tissue, or needle electrodes, which penetrate the tissue. Voltage is applied across the electrodes to induce an electric field in the treatment area, causing damage to the cells in the treatment area. Current electrode assemblies that incorporate surface electrodes do not effectively distribute the electrical treatment to the treatment area between the electrodes. Needle electrodes more effectively distribute the electrical treatment; however, needle electrodes are more unpleasant for the patient.

### SUMMARY

To distribute electrical treatment to a treatment area of a patient (e.g., human or animal subject), one general aspect includes an electrode assembly for delivery of the electrical therapy. The electrode assembly may include a conductive spacer and at least two electrodes. Each electrode may include a conductive treatment surface configured to apply electrical treatment to a treatment area and a conductive non-treatment surface. The conductive non-treatment surface may be configured to contact the conductive spacer. The conductive spacer is positioned between the at least two of the electrodes and configured to electrically contact a surface of the treatment area between the electrodes.

The electrode assembly of the present disclosure may be implemented as a hand-held device, as a catheter, or other percutaneously delivered device. Implementations may include various combinations of the following features. In some embodiments, the conductive spacer may be a hydrogel, a conductive adhesive, a conductive gel, a conductive silicone, a urethane rubber, conductive thermoset, thermoplastic resins, any other biocompatible material with the desired conductivity, any semi-conductor material, or any combination thereof.

In some embodiments, a conductivity of the conductive spacer is substantially equal to ten times (10×) a conductivity of a tissue of the treatment area.

In some embodiments, a conductivity of the conductive spacer is greater than or equal to a conductivity of a tissue of the treatment area and less than or equal to one hundred times (100×) the conductivity of the tissue of the treatment area.

In some embodiments, a height of the conductive spacer is based on a distance between the electrodes.

In some embodiments, a height of the conductive spacer is greater than or equal to twenty percent (20%) of a distance between the electrodes and less than or equal to fifty percent (50%) of the distance between the electrodes. In some embodiments, a height of the conductive spacer is up to 70% of a distance between the at least two electrodes. In some embodiments, the optimal height may be between 20% and 60% of the distance, including for example, between 30% and 50% of the distance between the at least two electrodes.

In some embodiments, the conductive treatment surface of each electrode electrically contacts the treatment area, for example, indirectly via a gel that helps ensure electrical contact between the treatment area and the electrode. In some embodiments, the conductive treatment surface of each electrode directly contacts the treatment area, for example, with surface electrodes touching the surface of the treatment area or with penetrating electrodes puncturing and entering the treatment area.

In some embodiments, the electrodes are bipolar or monopolar electrodes.

In some embodiments, at least one or more of the electrodes are surface electrodes. In some embodiments, at least one or more of the electrodes are penetrating electrodes, such as needle electrodes, blade electrodes, or knife electrodes. In some examples, the at least one or more of the electrodes comprise a combination of non-penetrating (e.g., surface) and penetrating electrodes.

In some embodiments, a length of the conductive spacer is substantially equal to a length of at least one of the electrodes.

In some embodiments, the electrodes may include two rows of needle electrodes, and a length of the conductive spacer is substantially equal to a length of one or both of the two rows.

In some embodiments, the conductive spacer includes at least two conductive zones. Each of the conductive zones may have a different conductivity.

In some embodiments, the conductive spacer may include a recess, for example, a geometric cutout. In yet further implementations and examples, the conductive spacer may comprise a semi-conductive material and may be configured to act/function as both: the conductive spacer and a resistor. In various examples, the conductive spacer may have a conductivity or geometry, or both, configured to allow the conductive spacer to function also as a resistor. For example, in any of the embodiments and examples of the electrode assemblies, treatment tips, electrical therapy devices and/or methods of the present disclosure, the conductive spacer may be configured to act as a parallel resistor, for example, to match an impedance of an electrode assembly to an impedance of a pulse generator that generates and applies electrical energy via the electrode assembly. The conductive spacer may be configured, for example, to reduce an impedance of the electrode assembly.

According to one general aspect of the present disclosure, an electrical therapy device is provided. The device may comprise a plurality of exchangeable electrode assemblies and a treatment applicator configured to be coupled to one of the plurality of exchangeable electrode assemblies. Each of the plurality of electrode assemblies may include at least two electrodes each having a conductive treatment surface configured to apply electrical treatment to a treatment area of a subject and a conductive non-treatment surface configured to contact a conductive spacer. The conductive spacer may be disposed between the at least two electrodes, wherein the conductive spacer is configured to electrically contact a surface of the treatment area of the subject between the at least two electrodes. In some examples and implementations the conductive spacer may be configured to play a dual role as both a conductive spacer and also a resistor. Such conductive spacer/resistor may comprise a semi-conductive material having conductivity to achieve both of these two functions.

Another general aspect includes a method of administering electrical therapy, for example, to a patient. The method of administering electrical therapy may include in some implementations positioning an electrode assembly that includes at least two electrodes and a conductive spacer disposed between two of the electrodes on a treatment area such that a conductive treatment surface of each of the electrodes electrically contacts the treatment area, and the conductive spacer electrically contacts a surface of the treatment area between the electrodes. The therapy also includes applying a voltage to the treatment area via the electrodes. In some examples the administering electrical therapy may be for cosmetic indications, for example, to improve appearance of the treatment area.

Some embodiments of this aspect may include one or more of the following features. In some embodiments, the method may include selecting the electrode assembly. Selecting the electrode assembly may be based in some embodiments on the conductive spacer having a conductivity that is substantially equal to ten times (10×) a conductivity of a tissue of the treatment area (e.g., a surface of the treatment area in some applications).

In some embodiments, selecting the electrode assembly may be based on the conductive spacer having a conductivity that is less than, greater than, or equal to a conductivity of a tissue of the treatment area. In some embodiments, the conductivity may be greater than or equal to the conductivity of the tissue of the treatment area and less than or equal to one hundred times (100×) the conductivity of the tissue of the treatment area.

In some embodiments, selecting the electrode assembly is further based at least in part on a size of the electrode assembly and/or a size of the treatment area.

In some embodiments, the method may include placing a conductive gel on the surface of the treatment area to encourage contact between the conductive treatment surface of each of the at least two electrodes with the surface of the treatment area, the conductive spacer with the surface of the treatment area, or both.

In some embodiments, the method may include selecting the voltage to be applied to the treatment area based at least in part on a conductivity of the conductive spacer. In some embodiments, the method may include selecting the electrode assembly based at least in part on a conductivity of the conductive spacer. In various examples, the method may include using the conductive spacer as a resistor to match an impedance of an electrode assembly to an impedance of a pulse generator that generates and applies electrical energy via the exchangeable electrode assembly. In some examples, the method may include using the conductive spacer to reduce an impedance of the electrode assembly. In some examples, the method may comprise selecting the electrode assembly and/or conductive spacer based on a conductivity of a material, or geometry, or both the conductivity of the material and a geometry, of the conductive spacer. Also, the conductivity of the material and/or geometry of the conductive spacer may be selected to improve a treatment depth, or a value of the resistance, or both. In various examples, the method may comprise selecting a shape of the conductive spacer to change a shape of the treatment zone or area. The method may also comprise using a shape of the conductive material to change a value of the parallel resistance.

Yet another general aspect of the present disclosure includes an electrical therapy device. According to some examples, the electrical therapy device includes a treatment applicator configured to be coupled to an exchangeable electrode assembly (such exchangeable electrode assembly may be also referred to, for example, as a treatment tip), and in some examples may further include a display device. The exchangeable electrode assemblies are each configured to be coupled to and removed from the treatment applicator. Each exchangeable electrode assembly includes at least two electrodes and a conductive spacer positioned between the electrodes for applying an electrical therapy to a treatment area of a subject (e.g., patient). The electrical therapy device also includes a controller configured to execute instructions that, upon execution cause the controller to determine or receive an indication of the exchangeable electrode assembly coupled to the treatment applicator and may, in some examples, optionally cause displaying configuration information on the display device based on the conductive spacer of the exchangeable electrode assembly. Other modes of notifying a user of the configuration information may be used instead of or in addition to displaying. In some examples, the controller may be further configured to provide electrical treatment parameters for a treatment area based, at least in part, on the indication of the one of the plurality of exchangeable electrode assemblies.

In some embodiments of the electrical therapy device, the configuration information includes a type of tissue suitable for treatment with the exchangeable electrode assembly.

Another general aspect includes a method of selecting an electrical therapy device. The method may include selecting an electrode assembly based at least in part on a conductive spacer of the electrode assembly, where the electrode assembly includes at least two electrodes, two of which conductively contact the conductive spacer. The method may further include positioning the electrode assembly within a treatment area such that a conductive portion of each of the electrodes and a conductive surface of the conductive spacer contact the treatment area.

According to another general aspect of the present disclosure a method of operation of an electrical therapy device is provided. The method comprises determining or receiving an indication of an electrode assembly coupled to a treatment applicator, the electrode assembly comprises at least two electrodes that conductively contact a conductive spacer; determining characteristics of the conductive spacer based, at least in part, on the indication of the electrode assembly; and selecting treatment configuration information based, at least in part, on the electrode assembly and the characteristics of the conductive spacer. In some implementations, selecting treatment configuration may comprise selecting treatment parameters and the method may comprise selecting one or more of the following: a type of tissue to be treated with the electrode assembly, a treatment duration, a treatment depth, a voltage field, pulse duration, pulse frequency, a number of pulses. In some examples, the indication of the electrode assembly may include at least one of the following: a size of the electrode assembly, a type of the electrode assembly, a number of the electrodes, or a model number of the electrode assembly. In some examples, the method may further comprise using the conductive spacer as the resistor based on either a conductivity of a material of the conductive spacer or a geometry of the conductive spacer, or both the conductivity and the geometry. In yet further examples, the method may include displaying treatment configuration information on a display device.

In various examples, the method may include using the conductive spacer as a resistor to match an impedance of an electrode assembly to an impedance of a pulse generator that generates and applies electrical energy via the electrode assembly. In some examples, the method may include using the conductive spacer to reduce an impedance of the electrode assembly. In some examples, the method may comprise selecting the electrode assembly and/or conductive spacer based on a conductivity of a material, or geometry, or both the conductivity of the material and a geometry, of the conductive spacer. Also, the conductivity of the material and/or geometry of the conductive spacer may be selected to improve a treatment depth, or a value of the resistance, or both. In some examples, the method may comprise selecting a shape of the conductive spacer to change a shape of a treatment zone or area. In some examples, the method may also comprise using a shape of the conductive material to change a value of the parallel resistance. In any of the methods of the present disclosure the method may comprise using the conductive spacer also as a resistor by selecting the electrode assembly and/or the conductive spacer to optimize both a desired resistance value and a treatment depth.

Other features and advantages of the devices and methods of the present disclosure will become apparent from the following detailed description of various implementations.

### BRIEF DESCRIPTION OF THE DRAWINGS

FIG. 1 illustrates a nanosecond pulse generator apparatus in accordance with some embodiments.

FIG. 2 illustrates an electrode assembly having surface electrodes in accordance with some embodiments.

FIGS. 3A and 3B illustrate an exposed view of a portion of some examples of the configuration of the electrode assembly of FIG. 2.

FIG. 4A illustrates an exchangeable electrode assembly in accordance with some embodiments.

FIG. 4B illustrates a electrode assembly having needle electrodes in accordance with some embodiments.

FIG. 4C illustrates a proximal end view of an exchangeable electrode assembly in accordance with some embodiments.

FIG. 5A illustrates an exchangeable electrode assembly before coupling with a handle portion of a treatment applicator in accordance with some embodiments.

FIG. 5B illustrates the exchangeable electrode assembly of FIG. 5A after coupling with the handle portion.

FIG. 6A illustrates an exemplary balloon catheter electrode assembly in accordance with some embodiments. FIGS. 6B and 6C illustrate an example of a percutaneous needle electrode assembly.

FIGS. 7A and 7B illustrate cross-sectional views of examples of electrode assemblies having surface electrodes in accordance with some embodiments.

FIGS. 8A and 8B illustrate cross-sectional views of examples of electrode assemblies having needle electrodes in accordance with some embodiments.

FIGS. 9A and 9B illustrate cross-sectional views of examples of electrode assemblies having needle electrodes and center surface electrodes in accordance with some embodiments.

FIG. 9C illustrates a perspective view of an example electrode assembly having needle electrodes and center surface electrodes in accordance with some embodiments.

FIG. 10A illustrates a method for performing electrical therapy in accordance with some embodiments.

FIG. 10B illustrates a method for selecting an electrode assembly and parameters for an electrical treatment in accordance with some embodiments.

FIG. 11 illustrates a method for configuring a pulse generator to perform electrical therapy in accordance with some embodiments.

FIG. 12A illustrates an exemplary model of a treatment area of a surface electrode assembly having an insulative spacer.

FIG. 12B illustrates an exemplary model of a treatment area of a surface electrode assembly having a conductive spacer in accordance with some embodiments.

FIG. 13A illustrates an exemplary model of a treatment area of another surface electrode assembly having an insulative spacer.

FIG. 13B illustrates an exemplary model of a treatment area of another surface electrode assembly having a conductive spacer in accordance with some embodiments.

FIG. 14A illustrates an exemplary model of a treatment area for a surface electrode assembly having a conductive spacer having a height that is 10% of the width in accordance with some embodiments.

FIG. 14B illustrates an exemplary model of a treatment area for a surface electrode assembly having a conductive spacer having a height that is 20% of the width in accordance with some embodiments.

FIG. 14C illustrates an exemplary model of a treatment area for a surface electrode assembly having a conductive spacer having a height that is 30% of the width in accordance with some embodiments.

FIG. 14D illustrates an exemplary model of a treatment area for a surface electrode assembly having a conductive spacer having a height that is 40% of the width in accordance with some embodiments.

FIG. 14E illustrates an exemplary model of a treatment area for a surface electrode assembly having a conductive spacer having a height that is 50% of the width in accordance with some embodiments.

FIG. 15 illustrates a graph of value changes as the conductive spacer height is increased in accordance with some embodiments.

FIG. 16A illustrates a model of a treatment area for a surface electrode assembly having a conductive spacer with conductivity similar to the conductivity of the treatment area in accordance with some embodiments.

FIG. 16B illustrates a model of a treatment area for a surface electrode assembly having a conductive spacer with conductivity of approximately 10 times of conductivity of the treatment area in accordance with some embodiments.

FIG. 16C illustrates a model of a treatment area for a surface electrode assembly having a conductive spacer with conductivity of approximately 100 times of conductivity of the treatment area in accordance with some embodiments.

FIG. 16D illustrates a model of a treatment area for a surface electrode assembly having a conductive spacer with conductivity of approximately 1000 times of conductivity of the treatment area in accordance with some embodiments.

FIG. 17 illustrates a graph of value changes as the conductive spacer conductivity is increased in accordance with some embodiments.

FIG. 18A illustrates a model of a treatment area for a three-row needle electrode assembly having an insulative spacer.

FIG. 18B illustrates a model of a treatment area for a three-row needle electrode assembly having a conductive spacer in accordance with some embodiments.

FIG. 18C illustrates a model of a treatment area for an electrode assembly with two-rows of needle electrodes and a center surface electrode and having an insulative spacer.

FIG. 18D illustrates a model of a treatment area for an electrode assembly with two-rows of needle electrodes and a center surface electrode and having a conductive spacer in accordance with some embodiments.

FIG. 19A illustrates a model of a treatment area for an electrode assembly with surface electrodes having a conductive spacer with two conductivity zones, one being 10 times the other, in accordance with some embodiments.

FIG. 19B illustrates a model of a treatment area for an electrode assembly with surface electrodes having a conductive spacer with two conductivity zones, one of which is insulative, in accordance with some embodiments.

FIG. 19C illustrates a model of a treatment area for an electrode assembly with surface electrodes having a conductive spacer with two different conductivity zones, in accordance with some embodiments.

FIG. 20A illustrates a model of a treatment area for an electrode assembly with three surface electrodes having conductive spacers with different conductivity zones between the electrodes in accordance with some embodiments.

FIG. 20B illustrates a model of a treatment area for an electrode assembly with needle and surface electrodes having conductive spacers with different conductivity zones in accordance with some embodiments.

FIGS. 21A-21E illustrate models of a treatment area for electrode assemblies having recesses of varying shapes in the conductive spacer.

FIGS. 22A and 22B illustrates models of a treatment area for electrode assemblies having strip electrodes positioned on conductive spacers in accordance with some embodiments.

FIG. 23A illustrates a model of a treatment area for an electrode assembly having a monopolar electrode.

FIG. 23B illustrates a model of a treatment area for an electrode assembly having a monopolar electrode with a conductive spacer, in accordance with some embodiments.

FIG. 24 illustrates a model of a treatment area for an electrode assembly having two electrodes with a conductive spacer in accordance with some embodiments.

FIG. 25 illustrates an exemplary cross-sectional view of an electrode assembly portion of a treatment applicator, where a conductive spacer in the electrode assembly is configured to act as the conductive spacer and also as a parallel resistor in accordance with some embodiments.

### DETAILED DESCRIPTION

It has been shown that electric therapy, such as nsPEF treatments can be used, among other things, to cause unwanted cells, such as tumor cells and lesions to undergo apoptosis, a programmed cell death. Tests have shown that such lesions and tumors can shrink to nonexistence after treatment. No drugs may be necessary. It has also been shown that the subject's immune system may be stimulated to attack all similar unwanted cells, such as tumor cells, including those of tumors that are not within the nsPEF-treated tumor.

A “tumor” includes any neoplasm or abnormal, unwanted growth of tissue on or within a subject. A tumor can include a collection of one or more cells exhibiting abnormal growth. There are many types of tumors. A malignant tumor is cancerous, a pre-malignant tumor is precancerous, and a benign tumor is noncancerous. Examples of tumors include a benign prostatic hyperplasia (BPH), uterine fibroid, psoriasis, seborrheic keratosis, warts, sebaceous hyperplasia, pancreatic carcinoma, liver carcinoma, kidney carcinoma, colon carcinoma, pre-basal cell carcinoma, melanoma, and tissue associated with Barrett's esophagus, just to name a few.

Sub-microsecond electric pulses, such as nanosecond pulses may be used for treatment of various conditions, disorders, and diseases, including but not limited to treatment of lesions, mucosal epithelium, gastrointestinal tract, esophagus, skin tumors and diseases, aged skin, abnormal tissue growth, cardiac conditions, such as atrial fibrillation, otolaryngological conditions.

Instruments, systems and methods of the present disclosure include application of short, high field strength electric pulses for the improved treatment of various conditions, disorders and diseases while minimizing or avoiding the risk of harming non-target tissue.

Pulse lengths of less than 1000 nanoseconds have been particularly studied to be effective in stimulating an immune response. Pulse lengths of about 10 to 1000 nanoseconds, are of particular interest in that they are long enough to carry sufficient energy to be effective at low pulse numbers but short enough to be effective in the manner desired.

In general, applying a treatment may comprise applying sub-microsecond electrical pulses. For example, applying sub-microsecond electrical pulses may comprise applying a train of electrical pulses having a pulse width of between 0.1 nanoseconds (ns) and 1000 nanoseconds (ns). In some variations, applying sub-microsecond electrical pulses comprises applying a train of nanosecond electrical pulses having peak voltages, for example, of less than 1 kilovolt per centimeter (kV/cm), between 1 kV/cm and 500 kV/cm, between 1 kV/cm and 100 kV/cm, or between 5 kV/cm and 50 kV/cm. Applying sub-microsecond electrical pulses may comprise applying a train of sub-microsecond electrical pulses at a frequency of between 0.1 (Hz) to 10,000 Hz. In other applications, other pulse frequencies and widths may be used. For example, microsecond or slower electrical pulses may be used. Further, the use of the conductive spacers described herein may be used in any electrical therapy treatment in which an improvement of the electric field between electrodes is desired. Accordingly, this disclosure is not limited to only pulsed technology but may also apply to, for example, radio frequency technology.

Various treatment instruments that include electrodes are known. In some of these instruments, multiple electrodes are used that are spaced apart. In others, an insulative spacer is placed between the electrodes. The insulative spacer may be used to avoid arcing and other issues that may arise from the current flowing in areas other than the tissue to be treated. While surface electrodes provide improved safety, less risk for infection, and are preferred by both physicians and patients over penetrating electrodes (e.g., needle electrodes, blade electrodes, knife electrodes) that must pierce the skin or other tissue being treated, surface electrodes with insulative spacers fail to provide sufficient treatment values at the desired depth in the treatment area between the electrodes. As such, needle electrodes are often more effective. However, for larger treatment areas, a larger electrode assembly is generally needed to treat the appropriate area. When the size of the electrode assembly is increased, the number of needle electrodes in the electrode assembly is increased. This increases the complexity and cost of manufacturing the electrode assemblies. Another issue with increasing the number of needle electrodes is that the required insertion force to drive all of the needles into the skin increases. As a result, it can become difficult to push the needles in, and the “bed of nails” phenomenon may occur in which the needles do not pierce the skin or otherwise poor needle penetration occurs. Additionally, even with the use of needle electrodes, it is difficult to effectively distribute the electric treatment in the treatment area.

To address these issues and provide a distributed effective treatment in the treatment area that reaches sufficient depth and may provide a safer alternative with fewer penetrating electrodes, a new electrode assembly is disclosed herein that includes a conductive spacer rather than an insulative spacer between the electrodes. Using such conductive spacer allows for more consistent treatment depth in the treatment area as well as limiting the need for needle electrodes. Further, even if needle electrodes are used, the conductive spacer can be used to reduce the need for additional rows of needles and/or a number of needles in a row. The conductive spacer in these treatments effectively prevents arcing between the electrodes and does not impede treatment, but rather improves treatment.

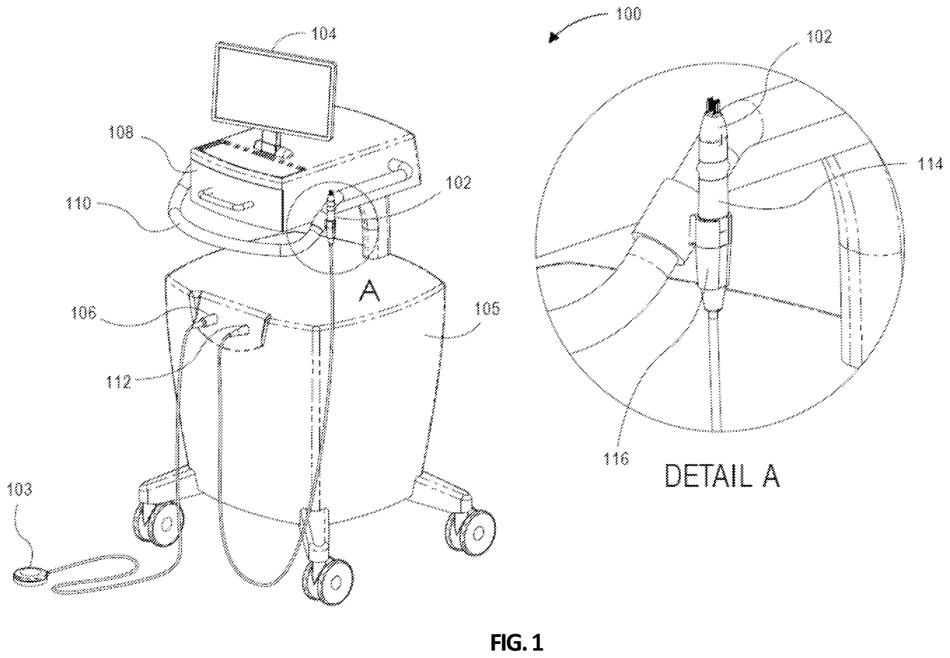

FIG. 1 illustrates a nanosecond pulse generator system in accordance with an embodiment. Any of the pulse generators as described in U.S. patent Ser. No. 10/548,665, filed May 6, 2016, entitled “HIGH-VOLTAGE ANALOG CIRCUIT PULSER WITH FEEDBACK CONTROL” and incorporated herein by reference may be used with the treatment apparatuses of the present disclosure. NsPEF system 100 includes treatment instrument or treatment applicator 102, footswitch 103, and interface 104. Footswitch 103 is connected to housing 105 and the electronic components therein through connector 106. Treatment applicator 102 is connected to housing 105 and the electronic components therein through high voltage connector 112. NsPEF system 100 also includes a handle 110 and storage drawer 108. As shown in DETAIL A portion of FIG. 1, nsPEF system 100 also includes holster 116, which is configured to hold treatment applicator 102 at its handle portion 114. The treatment applicator 102 includes an electrode assembly at the distal end which is described in more detail throughout this disclosure.

A human operator may input a number of pulses, amplitude, pulse duration, and frequency information, for example, into a numeric keypad or a touch screen of interface 104. The interface 104 may provide information regarding the treatment to the operator including providing configuration information for the user to view and adjust as well as treatment values and information during the administration of the treatment. In some embodiments, the pulse width can be varied. A controller 107 within housing 105 sends signals to pulse control elements within nsPEF system 100. The controller may be any suitable computing device that includes memory for storing instructions and a processor for executing the instructions. In some embodiments, fiber optic cables allow control signaling while also electrically isolating the contents of the metal cabinet with nsPEF generation system 100, the high voltage circuit, from the outside. To further isolate the system, system 100 may be battery powered instead of from a wall outlet.

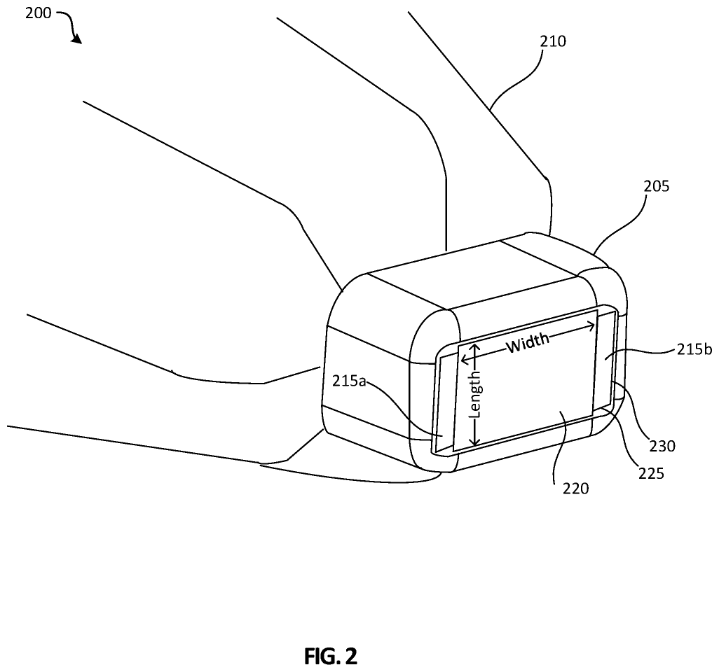

FIG. 2 illustrates a portion of an electrode assembly 200 in accordance with some embodiments. In some embodiments, the electrode assembly 200 may be coupled to a handle of a treatment applicator, such as treatment applicator 102. In some embodiments, electrode assemblies (such as the electrode assembly/treatment applicator 102) may be exchangeable such that different electrode assemblies may be removably coupled to the handle. The exchangeable electrode assemblies may be disposable (e.g., penetrating electrodes may be discarded after use) or may be reusable (e.g., surface electrodes may be reusable). In further embodiments the treatment applicator does not have a separate handle and the electrode assembly itself forms the treatment applicator or the electric treatment device. The electrode assembly 200 may include an insulative housing 205 that may be coupled to a housing 210. In some embodiments, the insulative housing 205 and housing 210 are a single piece of, for example, molded plastic. The insulative housing 205 may be made from any suitable insulative material, such as, for example, plastic. The electrode assembly 200 also includes two surface electrodes 215 (shown as 215a and 215b) and a conductive spacer 220.

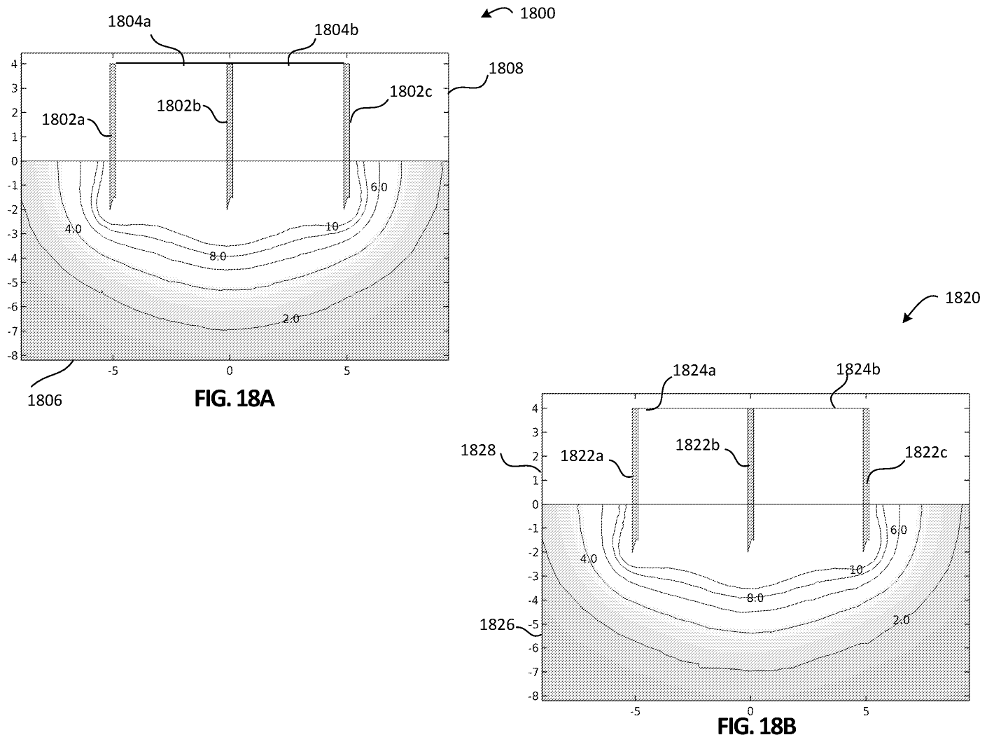

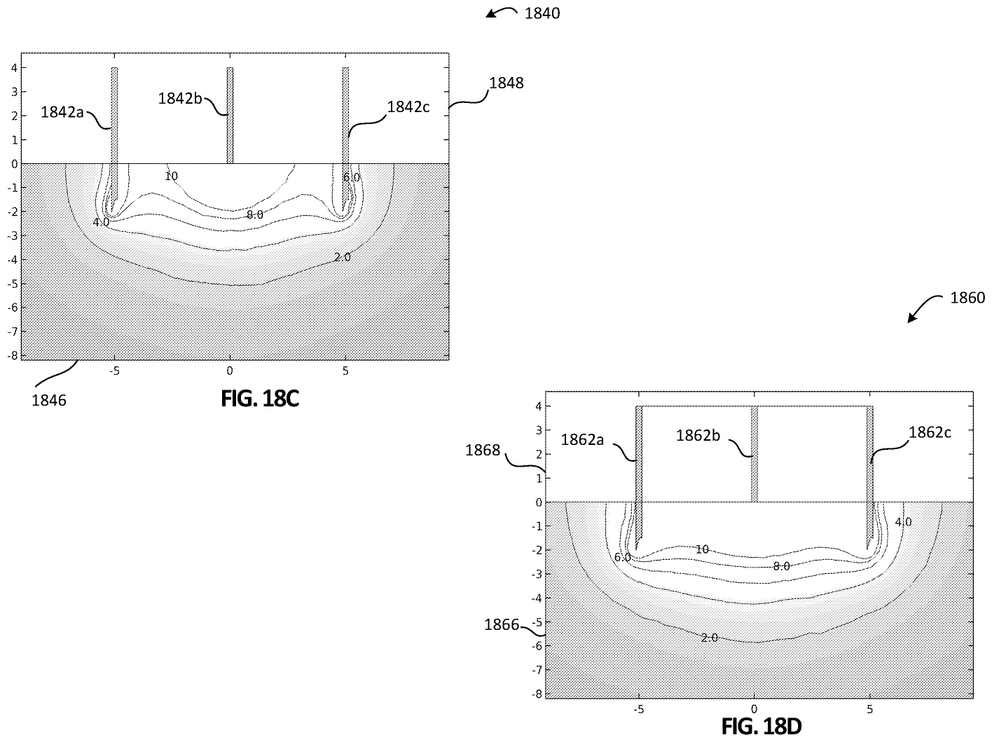

Two electrodes 215 are depicted, but electrode assembly 200 may include any number of electrodes. For example, as depicted in FIG. 18A, three rows of penetrating electrodes are included, and in other examples various numbers of electrodes may be provided. In some embodiments, the electrodes 215 are bipolar, such that one is negatively charged and the other is positively charged so that the current flows between the two electrodes 215. Bipolar configurations may be used with additional electrodes. Further, while FIG. 2 depicts two electrodes 215 enclosed within the same insulative housing 205 and housing 210, the electrodes 215 may each having their own housing. In some embodiments, the electrodes 215 may be monopolar electrodes, such that the current flows between the electrode and a return pad that is placed on the patient. The electrodes 215 may measure, for example, approximately one (1) millimeter wide (or thick) as measured on side 225 (the width of the electrode 215a or 215b) and, for example, five (5) millimeters long as measured on side 230 (the length of the electrodes 215a or 215b). Electrodes 215 may be made from any suitable conductive material such as, for example, stainless steel, graphite, noble metals (e.g., gold, silver, platinum), copper, titanium, brass, or any other suitable material. The electrodes 215 may have a height, which cannot be seen since it extends into insulative housing 205 in this view, but which will be shown in more detail in later figures. The electrodes 215 are positioned substantially in parallel with each other at a fixed distance apart. For example, in a 5-millimeter by 10-millimeter (5 mm×10 mm) electrode assembly, the electrodes 215 are positioned 10 mm apart and have a length of 5 mm. In a 5 mm×5 mm electrode assembly, the electrodes 215 are positioned 5 mm apart and have a length of 5 mm Dimensions of the electrodes may vary depending on the treatment application, including without limitations 1.5 mm by 1.5 mm, 10 mm by 10 mm, 10 mm by 1.5 mm, 20 mm by 1 mm, etc. Further, other configurations of electrodes 215 may include a ring electrode surrounding a center electrode, electrodes that are not evenly distributed, an array of electrodes, and the like. Additionally, while surface electrodes are depicted in FIG. 2, any type of electrode may be used including, for example, penetrating electrodes, catheter electrodes, clamp electrodes (e.g., parallel cardiac clamps or laparoscopic clamps), a Barrett's esophagus device, percutaneous needle electrodes, balloon electrodes, balloon catheter electrodes, and the like.

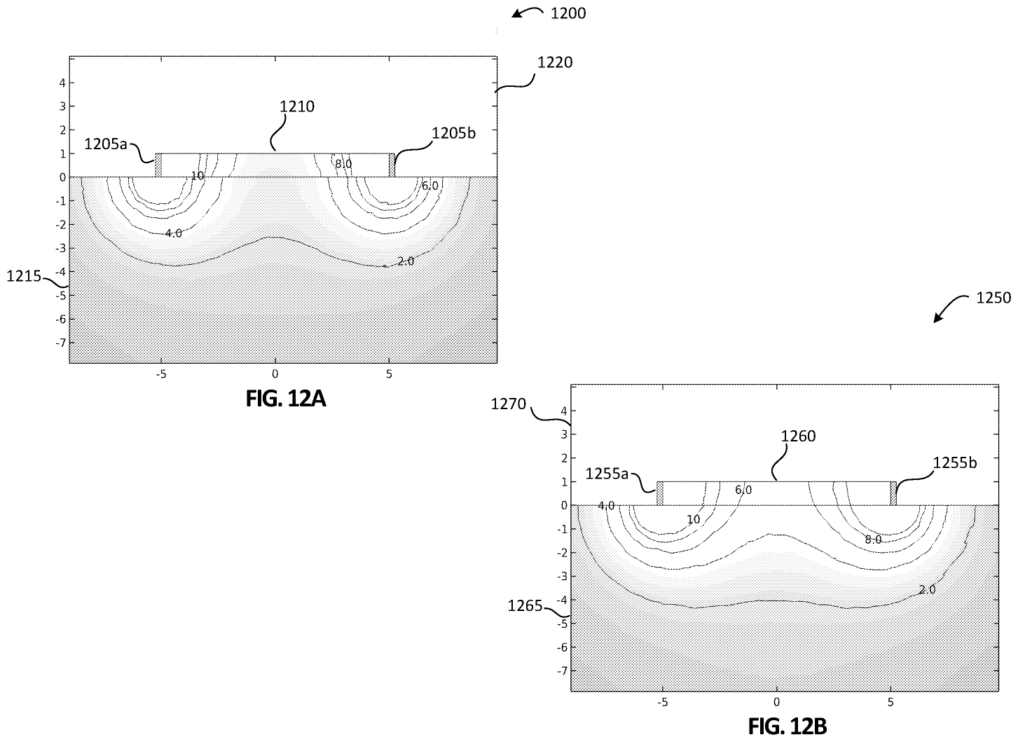

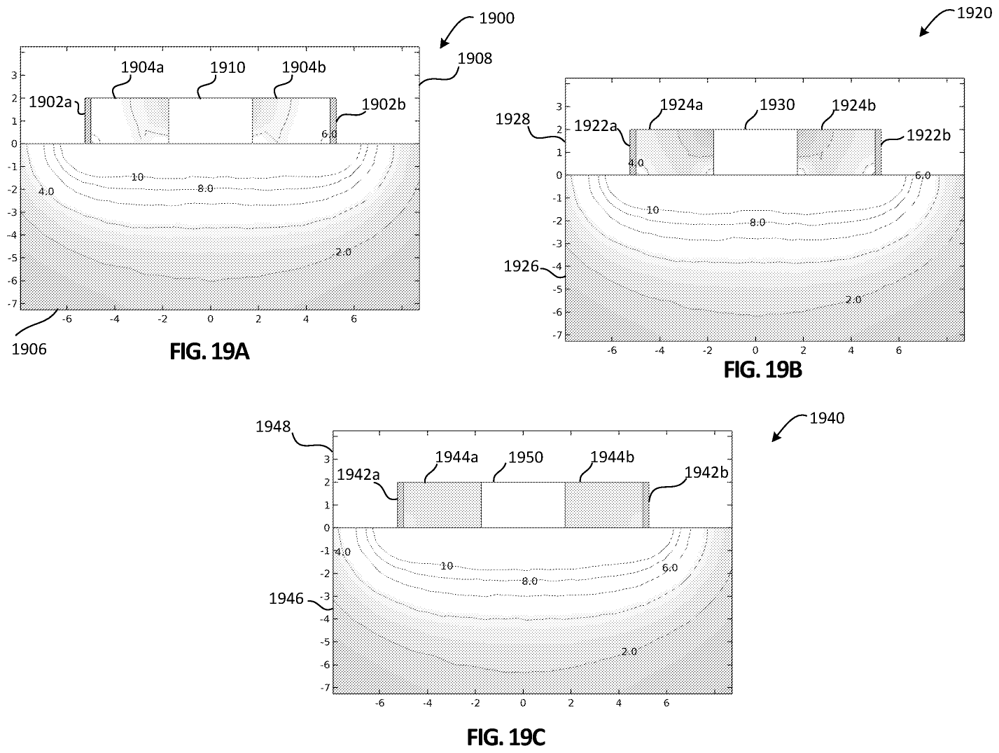

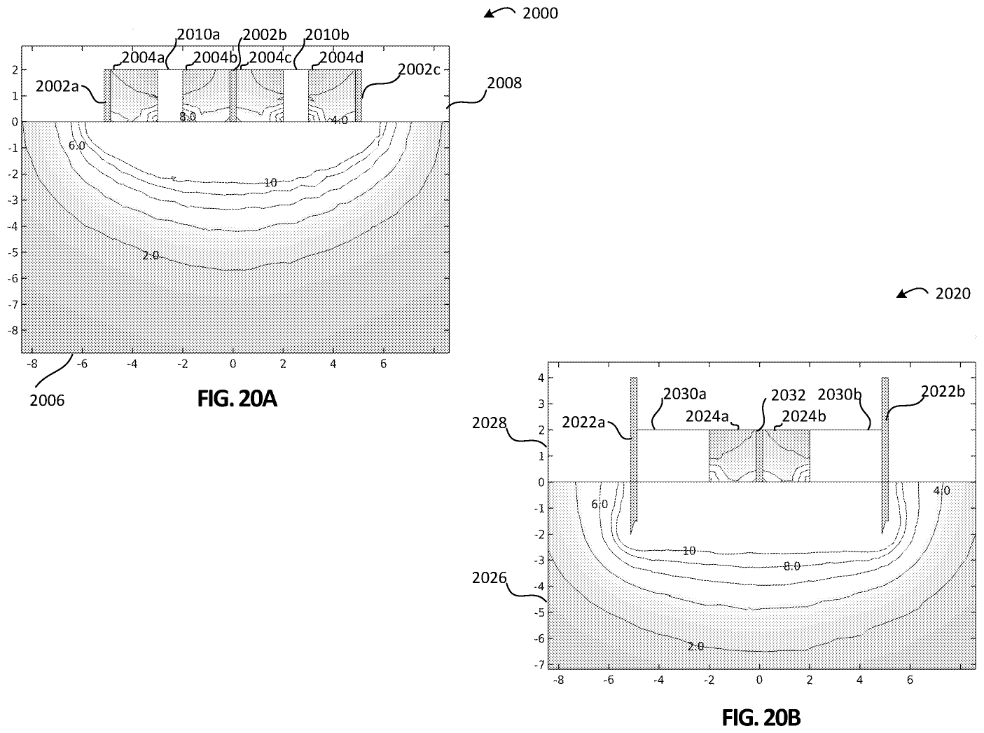

Conductive spacer 220 is positioned between electrodes 215a and 215b. Conductive spacer 220 may be made from any suitable conductive material having a conductivity that is desired for the conductive spacer 220. For example, conductive spacer 220 may be made from a hydrogel, a conductive adhesive, a conductive gel, a conductive silicone, a urethane rubber, carbon nanotubes, thermoplastic, thermoset resin, or any combination thereof. The desired conductivity of the conductive spacer 220 may be selected based on the conductivity of the skin or tissue being treated. For example, the conductivity of the conductive spacer 220 may be substantially the same conductivity as the treatment area of skin or tissue being treated up to approximately one hundred times (100×) the conductivity of the treatment area or tissue being treated. In some embodiments, the conductivity of the conductive spacer 220 may vary throughout the conductive spacer 220. For example, the conductive spacer 220 may have zones that have different conductivity and/or there may be a gradient of conductivity within the conductive spacer 220, such as in the examples shown in FIGS. 19A, 19B, 19C, 20A, and 20B. Various conductivity results are discussed with respect to FIGS. 12A-20B. The consistency of the conductive spacer 220 may be solid, compressible, or gelatinous yet firm enough to maintain shape and position within electrode assembly 200. The conductive spacer 220 may be in contact with the electrodes 215 such that it has a width of the distance between the two electrodes 215, or in some embodiments, the distance between two rows of electrodes (e.g., needle electrode rows). For example, if the electrodes 215 are spaced 10 mm apart, the width of the conductive spacer 220 is 10 mm. The length of the conductive spacer may be substantially similar to the length of the electrodes 215. For example, if the length of the electrodes 215 is 5 mm (as measured on side 230), the length of the conductive spacer 220 may also be 5 mm. The bottom of the conductive spacer 220 may be substantially flush with the bottom of the surface electrodes 215 such that both the conductive spacer 220 and the surface electrodes 215 contact the treatment area. In some embodiments, the conductive spacer 220 may extend into the insulative housing 205. In other words, the conductive spacer 220 and electrodes 215 may have conductive portions within the insulative housing 205 such that only a portion of the conductive spacer 220 and electrodes 215 are exposed.

The exposed surface of the conductive spacer 220 and the electrodes 215 is, in some embodiments, substantially flush with the opening of the insulative housing 205. Portions of the electrodes 215 that are not in contact with the conductive spacer 220 may be in contact with the insulative housing 205.

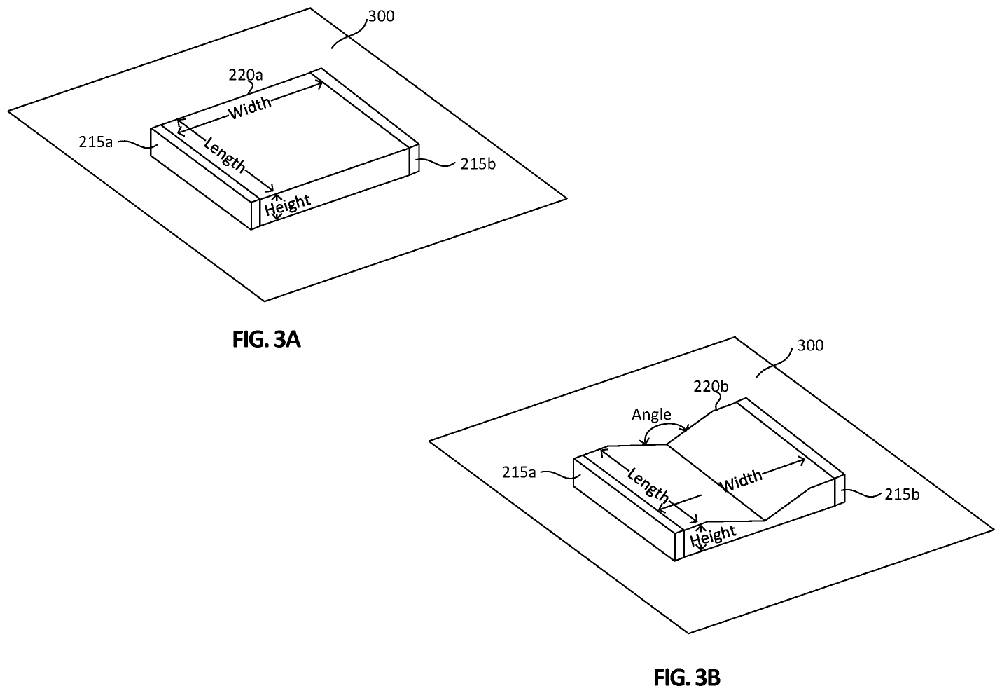

FIGS. 3A and 3B illustrate an exposed view of the electrodes 215 (215a and 215b) and conductive spacer 220 of electrode assembly 200. It was discovered that changing a shape or geometry of a conductive material such as conductive spacer between the electrodes, can help increasing e-field penetration and also the treatment zones can be evened out to become more uniform. As shown in FIG. 3A, the conductive spacer 220a is substantially the same height as the electrodes 215. In FIG. 3B, an alternative embodiment of conductive spacer 220b includes a recess or cutout, such that the height of the conductive spacer 220b varies with respect to the recess. In FIG. 3B, the recess is in a shape of a triangle having an angle, which may vary in various embodiments. The recess of the conductive spacer may be varied in shape and size (and it may be formed in some examples as a cutout of various geometric shapes) to affect a more evenly distributed treatment in some embodiments as is discussed in further detail herein. However, the shape of the recess is not limited to any geometric shape, and it can be formed in any suitable way and does not require cutting out a part of the material of the spacer. In some embodiments the electrodes 215 may extend further into an insulative housing such as insulative housing 205 or have insulative material wrapped around the electrodes 215, such that the electrically conductive exposed portion of electrodes 215 is the same height as the conductive spacer 220 that is in contact with the electrode 215. In some embodiments the electrodes 215 may be coupled to a conductor within insulative housing 205 or elsewhere within the electrode assembly 200. The height of the conductive spacer 220 may be selected based on, for example, the distance between the electrodes 215. For example, a height of the conductive spacer 220 that is approximately ten percent to fifty percent (10%-50%) of the distance between the electrodes 215 may be used. The distance between the electrodes 215 is substantially the same as the width of the conductive spacer 220, accordingly, the height of the conductive spacer 220 may be approximately 10%-50% of the width of the conductive spacer 220. A height of the conductive spacer 220 that is up to approximately forty percent (40%) the distance between the electrodes 215 may be used. For example, if the electrodes 215 are 10 mm apart such that the conductive spacer 220 has a width of 10 mm, the height of the conductive spacer 220 may be, for example, 4 mm. The conductive exposed portion of electrodes 215 that is in contact with the conductive spacer may also be 4 mm in height. The treatment surface of the conductive spacer 220 and the electrodes 215 may be placed on a surface of a treatment area 300 that may include skin or other tissue including heart, liver, or any other cell or tissue type to which treatment is to be applied.

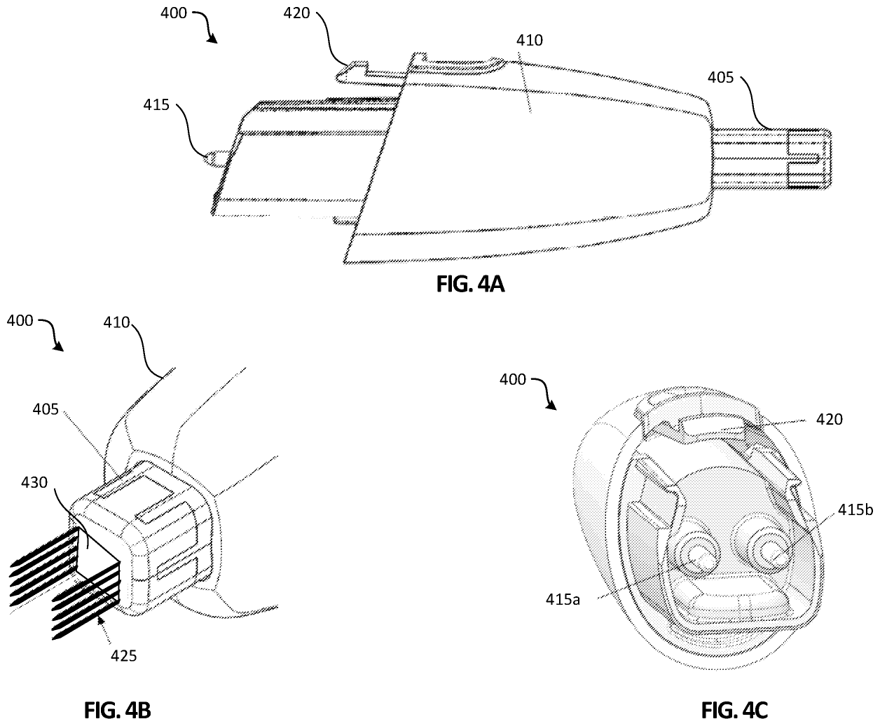

FIG. 4A illustrates a side view of an example of an exchangeable/removably coupled electrode assembly 400. Electrode assembly 400 includes housing 410 and insulative housing 405. The electrode assembly housing 410 may have a slightly elongated, tapered shape. Protruding from the distal end of the housing 410 is an insulative housing 405 that may include the electrodes (not shown in this view). Protruding from the proximal end of the electrode assembly 400 is a connector 415 that electrically connects the electrodes (not shown in this view) to the pulse generator to apply the electrical therapy to the patient via the electrodes. In this example, a mechanical connector 420 is used to mechanically attach the electrode assembly 400 on the proximal end to a handle of the treatment applicator.

FIG. 4B illustrates a view of the electrode assembly 400 having needle electrodes 425 and conductive spacer 430. The electrode assembly 400 may have a rectangular or square cross-section as shown, or may have any shape cross-section, such as a circle or oval. The needle electrodes 425 extend from the distal-facing (e.g., tissue-facing) end. The needle electrodes 425 may pierce the skin or tissue of the patient to apply the electrical treatment to the treatment area. The needle electrodes 425 may have a sharp and beveled distal end and be cylindrical needles in some embodiments. The conductive spacer 430 may be substantially the same as conductive spacer 220. Conductive spacer 430 contacts the needle electrodes 425. Conductive spacer 430 may have a height that extends into insulative housing 405 and/or housing 410, such that the height of the conductive spacer 430 is in contact with an exposed conductive surface of needle electrodes 425. The needle electrodes 425 may enter the tissue until the conductive spacer 220 is in contact with the surface of the treatment area (e.g., skin or tissue). Exchangeable electrode assembly 400 depicts needle electrodes 425, but plate or surface electrodes, such as surface electrodes 215, or a combination of surface and needle electrodes may be used in the exchangeable electrode assemblies 400 as described throughout this disclosure.

FIG. 4C illustrates the proximal end of the exchangeable electrode assembly 400. The mechanical connector 420 may snap or latch onto a handle (e.g., handle 114 in FIG. 1) of the treatment applicator (e.g., treatment applicator 102). The mechanical connector 420 may be a portion of the housing 410. The two electrical connectors 415 (shown as 415a and 415b) may electrically couple with an electrical connector in the handle. This proximal end may couple with the handle to make both a mechanical and electrical connection between the exchangeable electrode assembly 400 and the handle.

Within the exchangeable electrode assembly housing 410, in some embodiments, the electrodes may form part of an electrode assembly that is coupled to the electrode assembly housing 410 so that the electrodes are locked in position relative to the insulative housing 405 and electrode assembly housing 410.

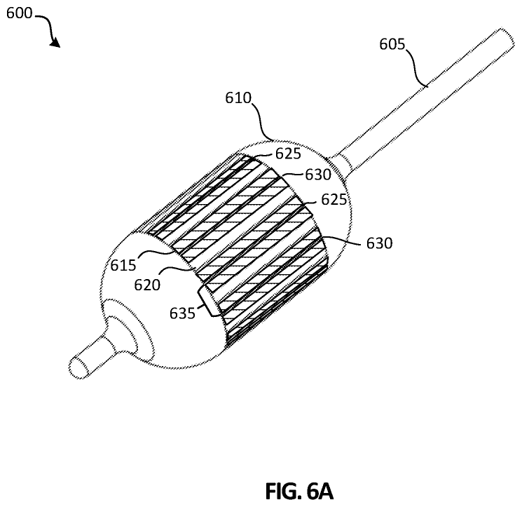

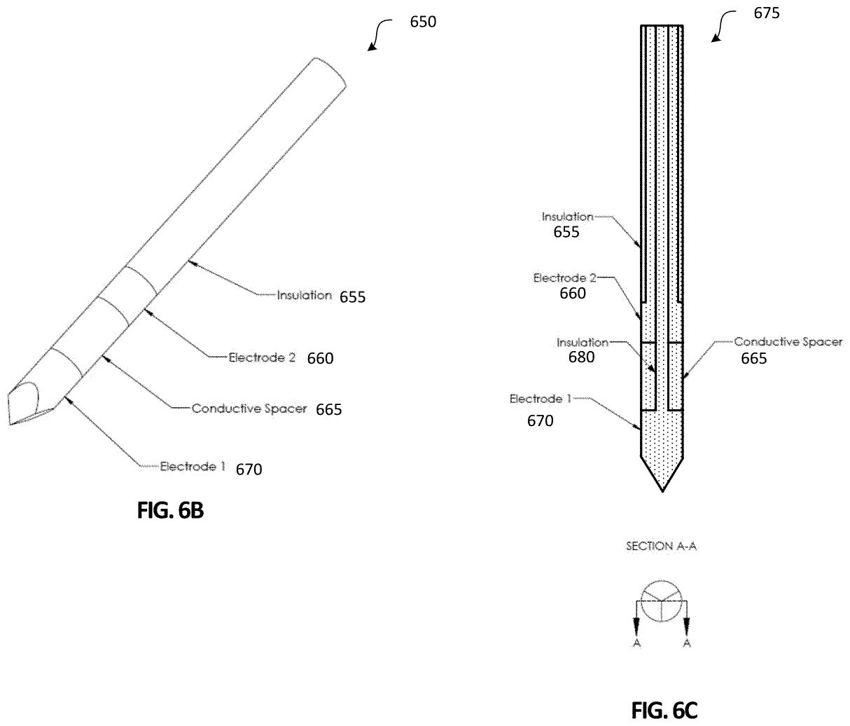

The electrode assemblies described herein may come in a variety of different sizes and configurations that may be used in multiple indications. For example, the size (e.g., diameter) of the treatment area on the distal face of the apparatus may be varied (e.g., between about 1 mm to 150 mm or more), and may be any appropriate shape (e.g., rectangular, rounded, triangular, oval, etc.). The electrodes used may be penetrating electrodes, surface electrodes, or a combination thereof. The conductive surface of the electrodes that contacts the treatment area may be any suitable size and will be discussed in more detail with respect to FIGS. 6-8. Portions of the electrodes may be surrounded by insulative material, and portions of the electrodes may be in contact with the conductive spacer. The portion of the electrodes in contact with the conductive spacer, that extend from the insulative housing, and the like, may all be adjusted as needed to apply treatment as described herein.

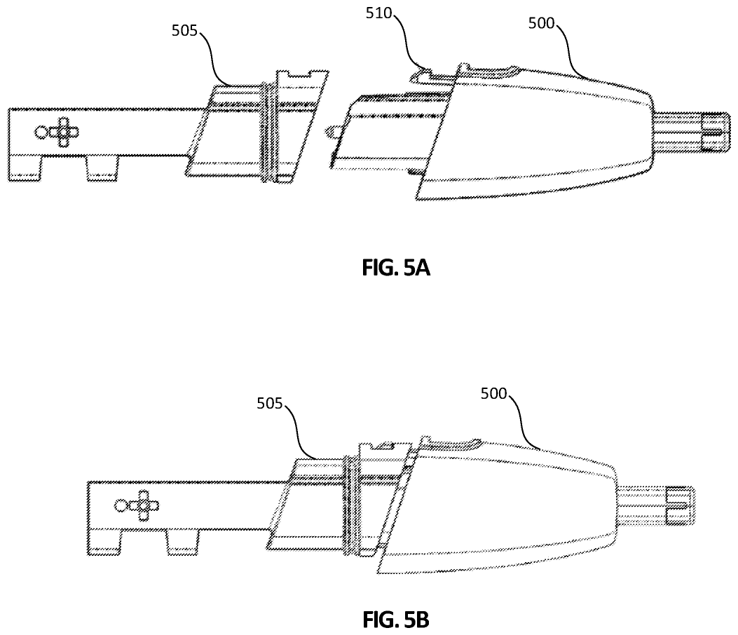

The exchangeable electrode assembly (e.g., a disposable or reusable electrode assembly) is generally configured to couple with a disposable or reusable handle. FIGS. 5A and 5B illustrate mechanical and electrical coupling between an exchangeable electrode assembly 500 and a portion of a handle 505. A connector 510 (shown by example as a clip—mechanical connector 420—in FIGS. 4A and 4B) may mechanically and releasably secure the exchangeable electrode assembly 500 and the handle 505 together.

FIG. 6A illustrates another example of implementation of the electrode assembly with a conductive space for percutaneous applications. In this example, an electrode assembly 600 has a balloon catheter configuration. The electrode assembly 600 includes a catheter body 605, an inflatable balloon 610, positive electrodes 625, negative electrodes 630, conductive spacers 635 having a first zone 615 and a second zone 620. In electrode assembly 600, the positive electrodes 625 alternate from the negative electrodes 630, such that every other electrode is positive, and the remaining electrodes are negative. Between the positive electrodes 625 and the negative electrodes 630 is a conductive spacer 635 that includes a first zone 615, a second zone 620, and another first zone 615. The first zone 615 may have a first conductivity and the second zone 620 may have a second conductivity. In some embodiments, the second zone 620 may be an insulative zone.

FIG. 6B illustrates a further example useful in percutaneous applications, such as a percutaneous needle electrode assembly 650. The percutaneous needle electrode assembly 650 may include a first electrode 670, a second electrode 660, a conductive spacer 665, and insulation 655.

FIG. 6C illustrates a cross-sectional view 675 of the percutaneous needle electrode assembly 650. The cross-sectional view 675 shows that there is additional insulation 680 within the percutaneous needle electrode assembly 650. The first electrode 670 extends through the center of the percutaneous needle electrode assembly 650 to the tip and is pointed to puncture tissue. Above the first electrode 670 along the shaft of the percutaneous needle electrode assembly 650, the conductive spacer 665 is between the first electrode 670 and the second electrode 660. The second electrode 660 is exposed along the shaft of the percutaneous needle electrode assembly 650 above the conductive spacer 665. The second electrode 660 also extends through the percutaneous needle electrode assembly 650 but is buffered from the treatment area above the exposed portion by insulation 655 and is also buffered from the first electrode 670 through the shaft of the percutaneous needle electrode assembly 650 by insulation 680.

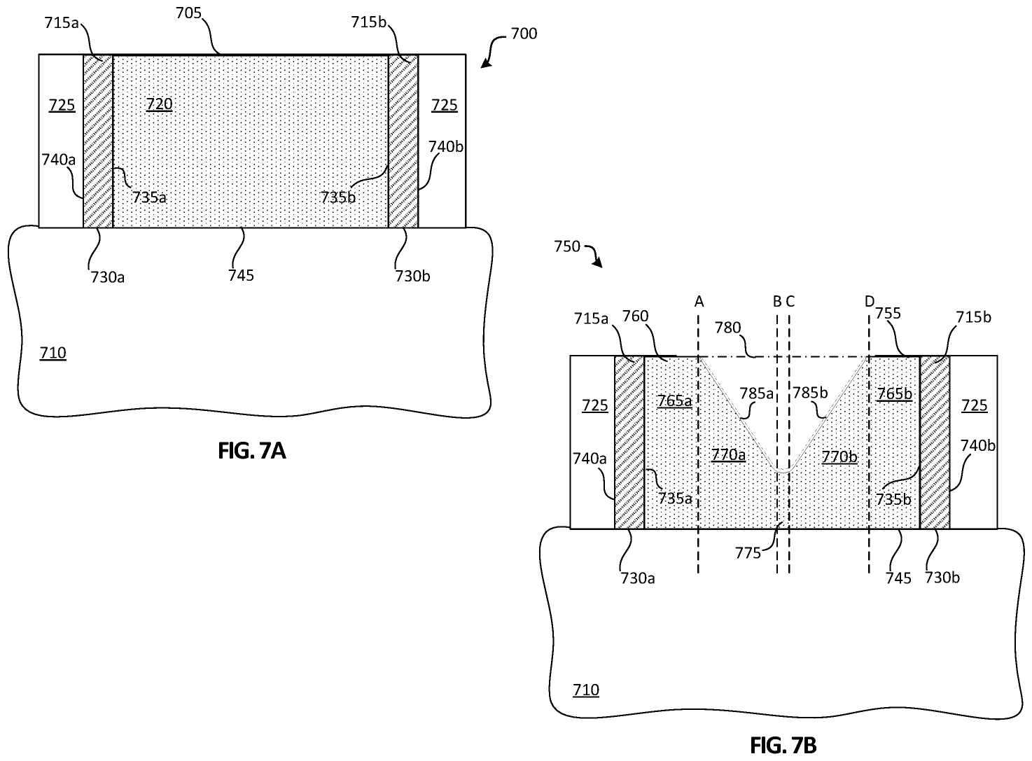

FIG. 7A illustrates a cross-sectional view 700 of a surface electrode assembly 705 applying treatment to treatment area 710. The treatment area 710 may be any suitable treatment area including a skin or tissue surface having, for example, a tumor.

The surface electrode assembly 705 may be similar to electrode assembly 200. The surface electrode assembly 705 may include surface electrodes 715 (shown by example as 715a and 715b), conductive spacer 720, and insulative housing 725. Surface electrodes 715 may be substantially the same as surface electrodes 215. Conductive spacer 720 may be the substantially the same as conductive spacer 220. Insulative housing 725 may be substantially the same as insulative housing 205. In some embodiments, the surface electrodes 715 and the conductive spacer 720 may extend out from the housing 725. In some embodiments, the surface electrodes 715 and the conductive spacer 720 may be recessed within the housing 725 such that contact with the treatment area 710 is achieved by drawing tissue from the treatment area 710 into the housing 725. Such configurations may be used throughout any of the described electrode assemblies herein.

As shown in cross-sectional view 700, a first surface 730a, 730b of each surface electrode 715a, 715b is in contact with the treatment area 710. The first surfaces 730 are a conductive treatment surface or portion of the electrodes 715. A second surface 735a, 735b of each surface electrode 715 is in contact with the conductive spacer 720. The second surfaces 735 are conductive non-treatment surfaces or portions of the electrodes 715. A third surface 740a, 740b of each surface electrode 715 is in contact with an insulative material such as insulative housing 725. A surface 745 of the conductive spacer 720, which is between the two electrodes 715, is in contact with a surface of the treatment area 710.

As shown, when a voltage is applied across the surface electrodes 715 (e.g., electrode 715a may have a positive charge and electrode 715b may have a negative charge), an energy pulse is applied to the treatment area 710. The conductive spacer 720 may serve to conduct some of the current from the energy pulse based on second surfaces 735 of surface electrodes 715 contacting conductive spacer 720. The conductivity of the materials allows the energy pulse to be directed to the treatment area 710 between the surface electrodes 715 and deeper into the treatment area 710. Such results are shown with respect to models depicted by example in FIGS. 12A-18D.

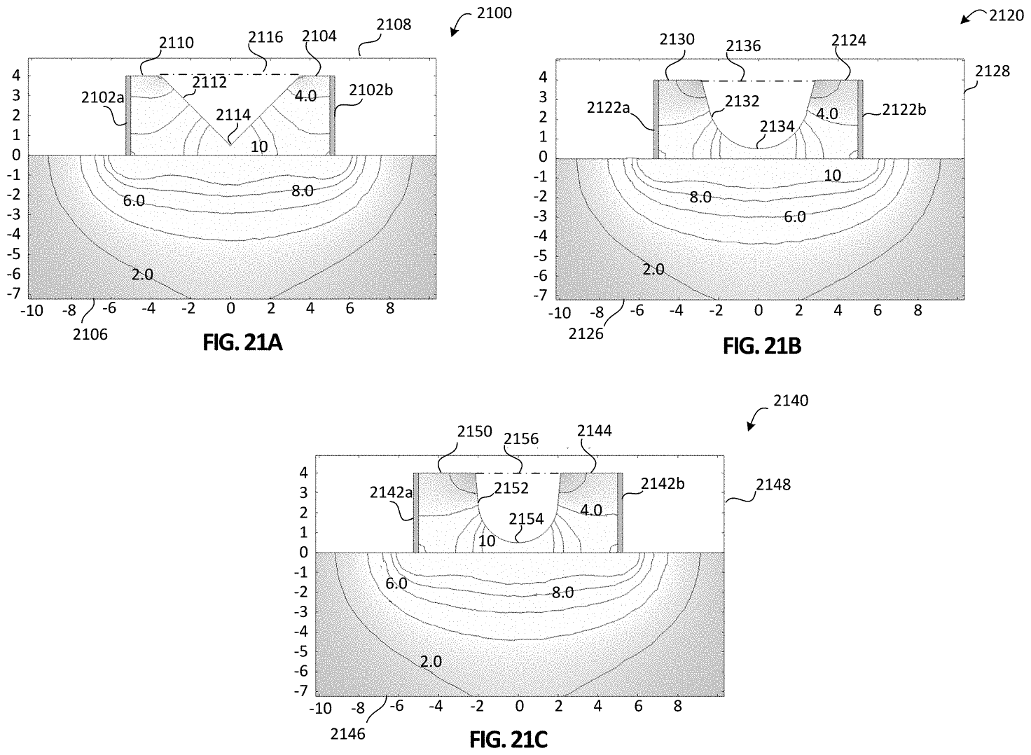

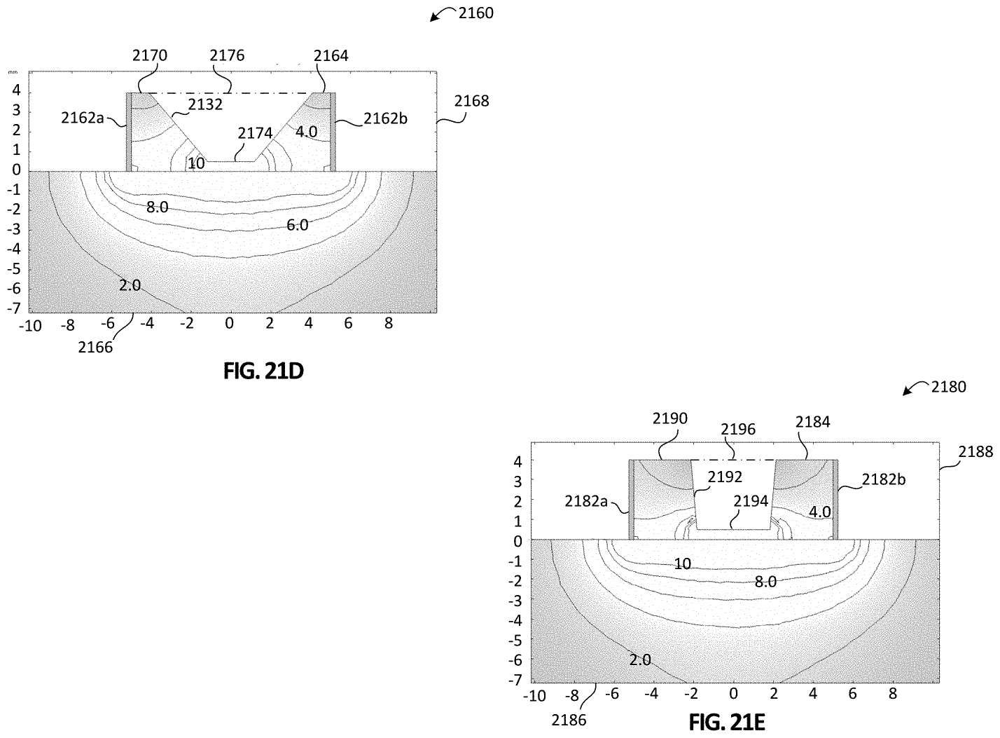

FIG. 7B illustrates an alternate cross-sectional view 750 of a surface electrode assembly 755 applying treatment to treatment area 710. In cross-sectional view 750, the conductive spacer 760 includes a recess 780. A surface 745 of the conductive spacer 760, which is between the two electrodes 715, is in contact with a surface of the treatment area 710. The conductive spacer 760 includes first portions 765, as shown. First portion 765a is disposed between the surface 735a of the electrode 715a and the dashed line “A,” and first portion 765b is disposed between surface 735b of the electrode 715b and the dashed line “D.” The first portion 765a has a first height such that the edge having the first height of the conductive spacer 760 is in contact with a conductive surface of electrodes 715. The conductive spacer 760 includes a second portion 775 between the dashed lines “B” and “C.” The second portion 775 has a second height lower than the first height. The second portion 775 may have an edge that is radial as shown in FIG. 7B, flat, such as depicted in FIG. 21D, or a point, such as depicted in FIG. 21A. The conductive spacer 760 includes third portions 770, each of which include angled edges 785 that extend from the first height at the first portion 765 to the second height at the second portion 775 and that defines the recess 780 (e.g., the “rounded V” cutout). As shown, third portion 770a has an angled edge 785a that extends from the first height of the first portion 765a to the second height at the second portion 775, and the third portion 770b has an angled edge 785b that extends from the first height of the first portion 765b to the second height at the second portion 775. The depicted recess 780 is exemplary such that any shape or size may be used to modify the shape of the conductive spacer 760.

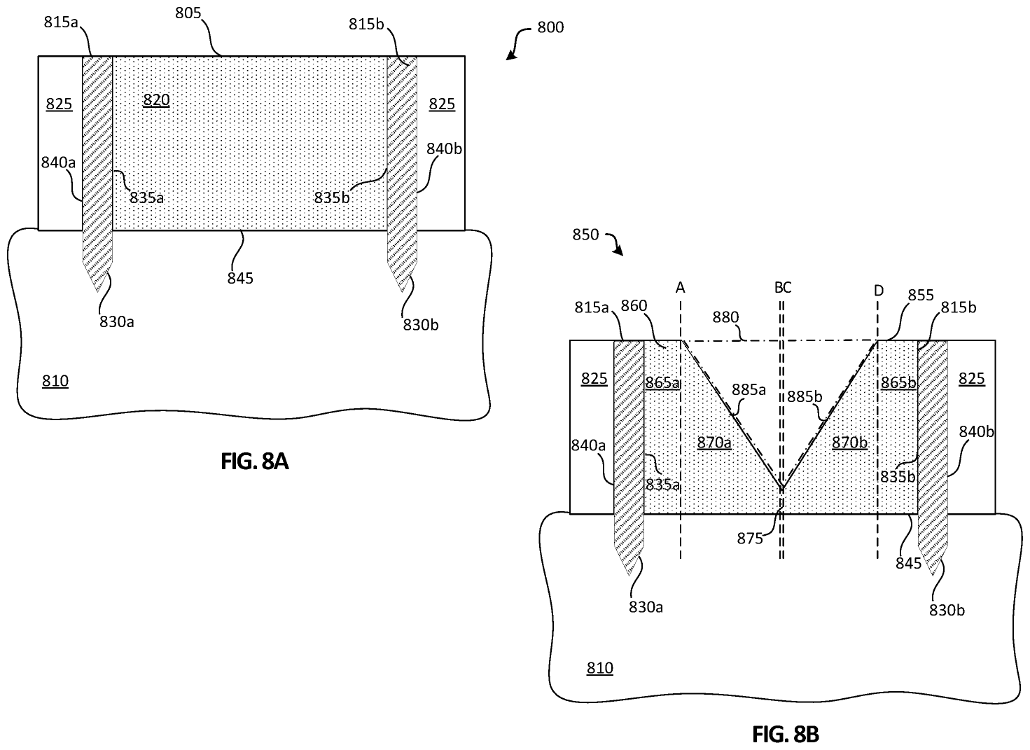

FIG. 8A illustrates a cross-sectional view 800 of an example of a needle electrode assembly 705 with penetrating electrodes applying electrical treatment to treatment area 810 of a patient. The treatment area 810 may be any suitable treatment area including a skin or tissue surface having, for example, a tumor, a lesion, or other unwanted condition.

The needle electrode assembly 805 may be similar to electrode assembly 400. The needle electrode assembly 805 may include needle electrodes 815, conductive spacer 820, and insulative housing 825. Needle electrodes 815 may be substantially the same as needle electrodes 425. Conductive spacer 820 may be the substantially the same as conductive spacer 430. Insulative housing 825 may be substantially similar to insulative housing 405.

As shown in cross-sectional view 800, a first surface 830a, 830b of each needle electrode 815a, 815b is in contact with the treatment area 810. The needle electrodes 815 may pierce the surface to extend within the treatment area 810. The first surfaces 830 are conductive treatment surfaces or portions of the electrodes 815 that are in contact with the treatment area 810. The second surfaces 835a, 835b of each needle electrode 815 are in contact with the conductive spacer 820. The second surfaces 835 are conductive non-treatment surfaces or portions of the electrodes 815. The third surfaces 840a, 840b of each needle electrode 815 may be in contact with an insulative material such as insulative housing 825 as illustrated in the example of FIG. 8A. A surface 845 of the conductive spacer 820, which is between the two electrodes 815, is in contact with a surface of the treatment area 810.

As shown, when a voltage is applied across the needle electrodes 815 (e.g., electrode 815a may have a positive charge and electrode 815b may have a negative charge), an energy pulse is applied to the treatment area 810. The conductive spacer 820 may serve to conduct some of the current from the energy pulse based on second surfaces 835 of needle electrodes 815 contacting conductive spacer 820. The conductivity of the materials allows the energy pulse to be directed to the treatment area 810 between the needle electrodes 815 and deeper into the treatment area 810. Such results are shown with respect to models depicted in FIGS. 12A-22B.

FIG. 8B illustrates an alternative cross-sectional view 850 of a needle electrode assembly with penetrating electrodes applying electrical treatment to treatment area 810 of a patient. In cross-sectional view 850, the conductive spacer 860 includes a recess 880. A surface 845 of the conductive spacer 860, which is between the two electrodes 815, is in contact with a surface of the treatment area 810. The conductive spacer 860 includes first portions 865, as shown. First portion 865a is disposed between the surface 835a of the electrode 815a and the dashed line “A,” and first portion 865b is disposed between surface 835b of the electrode 815b and the dashed line “D.” The first portions 865 have a first height such that the edge having the first height of the conductive spacer 860 is in contact with a conductive surface of electrodes 815. The conductive spacer 860 includes a second portion 875 between the dashed lines “B” and “C.” The second portion 875 has a second height lower than the first height. The second portion 875 may have an edge that comes to a point as shown in FIGS. 8B and 21A, flat, such as depicted in FIGS. 21D and 21E, or radial, such as depicted in FIGS. 21B and 21C. The conductive spacer 860 includes third portions 870, each of which include angled edges 885 that extend from the first height at the first portion 865 to the second height at the second portion 875 and that defines the recess 880 (e.g., the “V” cutout). As shown, third portion 870a has an angled edge 885a that extends from the first height of the first portion 865a to the second height at the second portion 875, and the third portion 870b has an angled edge 885b that extends from the first height of the first portion 865b to the second height at the second portion 875. The depicted recess 880 is exemplary such that any shape or size of the recess may be used to modify the shape the conductive spacer 860.

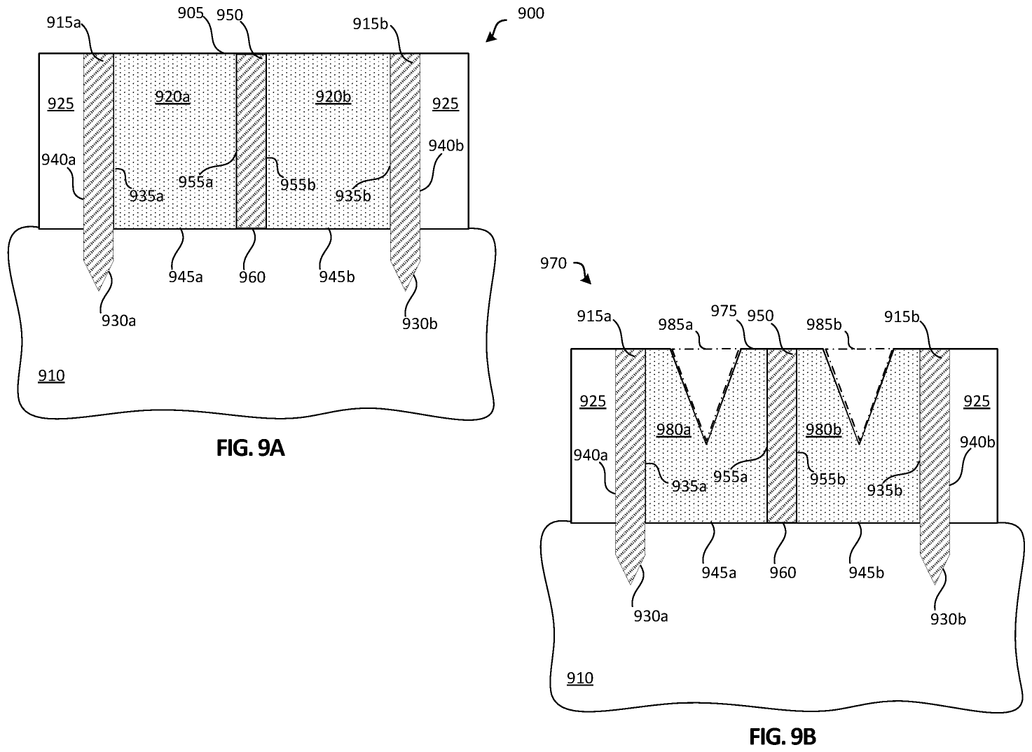

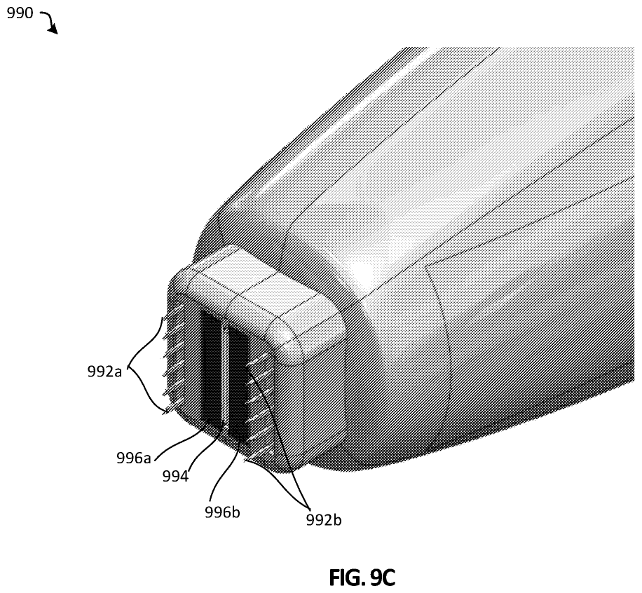

FIG. 9A illustrates an exemplary cross-sectional view 900 of a combination electrode assembly 905 applying treatment to treatment area 910 of a patient. The treatment area 910 may be any suitable treatment area including a skin or tissue surface having, for example, a tumor.

The combination electrode assembly 905 may include a plurality of needle electrodes 915 (shown by example as 915a and 915b), a center surface electrode 950, conductive spacers 920 (shown by example as 920a and 920b), and insulative housing 925. In some embodiments, the center electrode 950 may be a needle electrode and the outer electrodes 915 may be surface electrodes. Other combinations of electrodes may be included in a combination electrode assembly including penetrating, catheter, surface, or any other type of electrode. For example, in some embodiments the center electrode may be a needle electrode and the outer electrode may be a surface electrode in the shape of a ring that surrounds the center electrode. As another example, in some embodiments, the center electrode may be a surface electrode in the shape of a disk and the outer electrode may be a surface electrode in the shape of a ring that surrounds the center electrode. In yet other embodiments, the center and outer electrodes may be of the same type including penetrating, surface, catheter, or any other type of electrode. Needle electrodes 915a, 915b may be substantially the same as needle electrodes 425. Center surface electrode 950 may be substantially the same as surface electrodes 215. Conductive spacers 920a, 920b may be substantially the same as conductive spacer 220.

As shown in cross-sectional view 900, a first surface 930a, 930b of each needle electrode 915 is in contact with the treatment area 910. The needle electrodes 915 may pierce the surface to extend within the treatment area 910. The first surfaces 930 are conductive treatment surfaces or portions of the electrodes 915. A second surface 935a, 935b of each needle electrode 915 is in contact with the respective conductive spacers 920. For example, second surface 935a is in contact with conductive spacer 920a. Similarly, second surface 935b is in contact with conductive spacer 920b. The second surfaces 935 are conductive non-treatment surfaces or portions of the electrodes 915. Third surfaces 940a, 940b of each needle electrode 915 may be in contact with an insulative material such as insulative housing 925. Surfaces 945a, 945b of each conductive spacer 920 are in contact with a surface of the treatment area 910. Additionally, center surface electrode 950 includes a first surface 960 in contact with a surface of the treatment area 910 and second surfaces 955a, 955b, each in contact with the respective conductive spacers 920.

As shown, when a voltage is applied across the electrodes 915a, 915b, 950 (e.g., electrodes 915a and 915b may have a positive charge and electrode 950 may have a negative charge, or vice versa), an energy pulse is applied to the treatment area 910. The conductive spacers 920 may serve to conduct some of the current from the energy pulse based on second surfaces 935 of needle electrodes 915 contacting conductive spacers 920 and second surfaces 955 of surface electrode 950 contacting conductive spacers 920. The conductivity of the materials allows the treatment pulse to be directed to the treatment area 910 between the needle electrodes 915 and deeper into the treatment area 910. Such results are shown with respect to the models depicted in FIGS. 12A-22B.

FIG. 9B illustrates an alternative exemplary cross-sectional view 970 of a combination electrode assembly 975 applying treatment to treatment area 910. In cross-sectional view 970, the conductive spacers 980a, 980b include recesses 985a, 985b, respectively. Each conductive spacer 980 may be similar to conductive spacer 860 of FIG. 8B or conductive spacer 760 of FIG. 7B. The recesses 985 depicted in conductive spacers 980 are shown by example as a “V” cutout. However, any shape may be used, and in some embodiments the shape of each recess 985 may be different (e.g., recess 985a may be a “rounded V” shape and recess 985b may be “U”-shaped). The depicted recesses 985 are exemplary such that any shape or size may be used to modify the shape of the conductive spacers 980.

FIG. 9C illustrates a perspective view of an example electrode assembly 990 having needle electrodes 992, center surface electrode 994, and conductive spacers 996. In this example, the needle electrodes 992 include a row of electrodes 992a on one side of the electrode assembly 990 and a second row of electrodes 992b on the other side of the electrode assembly, and the surface electrode 994 (e.g., a plate 994) may be positioned in parallel between the two rows of electrodes 992a and 992b. The needle electrodes 992 may be substantially the same as the needle electrodes 915 as described with respect to FIGS. 9A and 9B. The surface electrode 994 may be substantially the same as the surface electrodes 950 described with respect to FIGS. 9A and 9B. The conductive spacers 996a and 996b may be substantially the same as conductive spacers 920 described with respect to FIG. 9A or conductive spacers 980 as described with respect to FIG. 9B.

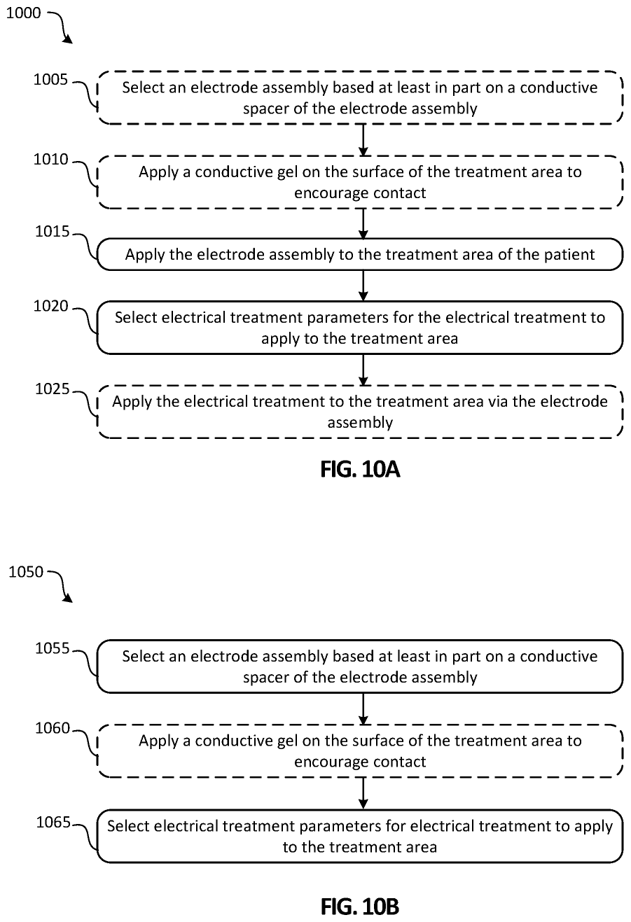

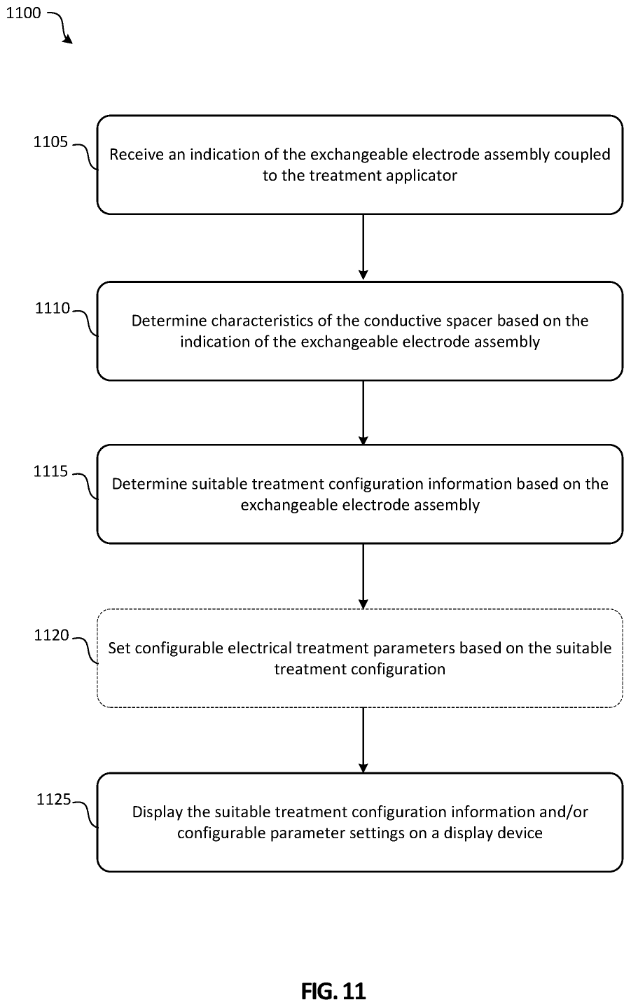

The disclosed treatment system with any of the electrode assemblies having conductive spacers as described above may be used to provide effective electrical treatment to a patient. FIGS. 10A, 10B, and 11 provide some examples of the process steps that can be selectively combined in various methods for selection of the electrode assemblies, selection of electrical treatment parameters, application of the electrical treatment, and display (or otherwise notifying/communicating) of the treatment information. In general, methods described herein, including the method illustrated in FIGS. 10A-10B and 11, may be for treatments of cosmetic indications, for example, to improve appearance of the treatment area. Such cosmetic indications typically have no symptoms other than the visible effects being treated and, while frustrating and may lead to negative psychosocial consequences, are generally without medical consequences. For example, a sebaceous hyperplasia (SH) is one example of a cosmetic indication. FIGS. 12A-22B provide models depicting electrical treatment distribution using various configurations of the electrode assemblies.

FIG. 10A illustrates a process 1000 for applying electrical treatment to a patient according to some embodiments. The process 1000 may be performed by a human user (e.g., a medical professional) operating the treatment device or by a system, for example, such as system 100 of FIG. 1. In some embodiments, at optional step 1005, the user may select an electrode assembly based at least in part on a conductive spacer of the electrode assembly. For example, the user may select an electrode assembly having a conductive spacer with a conductivity suitable for the tissue being treated. The conductivity of the conductive spacer may be more, less, or substantially equal to the conductivity of the treatment area. In some embodiments, the conductivity of the conductive spacer may be within a range such as greater than or equal to the conductivity of the treatment area and less than or equal to one hundred times (100×) the conductivity of the treatment area and may be, in some embodiments, slightly less than, or approximately equal, or approximately ten times (10×) the conductivity of the treatment area, or in some embodiments, approximately one to ten times (1×-10×) the conductivity of the treatment area. The conductivity of the treatment area may be determined based on a type of tissue being treated. For example, the treatment may be on skin, liver tissue, heart tissue, or any other type of tissue or cells. The conductivity of epidermis is approximately 1.1 S/m, so the range of conductivity of the conductive spacer may be from slightly less than 1.1 S/m to 110 S/m and may preferably be around 11 S/m (approximately 10 times the conductivity of the treatment area) in some embodiments. Treatment on other tissues may have different conductivity ranges as the conductivity of the tissue may vary with the type of tissue being treated. As another example, the user may select the electrode assembly having a conductive spacer with a recess that is desirable for the tissue being treated. For example, a recess having a second height (e.g., the valley of the “V” or “U” shape) that is smaller or larger, a steeper or less steep angle of the edge connecting the first height to the second height, a wider or narrower recess, or the like may have various benefits for treatment of various types of tissues. In yet another example, the controller of the pulse generator system (such as the one shown by example in FIG. 1) may receive information about treatment area and/or the tissue being treated and make a selection for the electrode assembly to use and provide such selection information to the user (e.g., technician or other medical personnel) for obtaining and using for the treatment. The electrode assembly may include at least two electrodes that each conductively contact the conductive spacer.

In some embodiments, at optional step 1010 the user may apply a conductive gel on the surface of the treatment area to encourage contact between the electrode assembly electrodes and the treatment area and/or between the conductive spacer of the electrode assembly and the treatment area. For example, conductive gels are often used to help encourage uniform contact of an electrode on skin or other treatment areas that are not precisely uniformly flat. The conductive gel differs from the conductive spacer of the electrode assembly in several ways. For example, the consistency of such conductive gel is typically much less viscous and firm than the conductive spacer. Further, the conductive gel is positioned between the surface of the conductive spacer and the surface of the treatment area and, in some surface electrode embodiments, between the surface of the surface electrode and the surface of the treatment area.

At step 1015, the user applies or positions the electrode assembly to the treatment area of the patient. For example, any surface electrodes and the conductive spacer are positioned to conductively contact (i.e., electrically contact) the surface of the treatment area. Any needle electrodes are inserted into the treatment area until the conductive spacer is pressed against and conductively contacting the surface of the treatment area. In electrode assemblies having electrodes and conductive spacer recessed in the housing, the tissue is drawn into the electrode assembly such that the tissue is in conductive contact with the electrodes and the conductive spacer.

At step 1020, the electrical treatment parameters are selected for the electrical treatment to apply to the treatment area. The electrical treatment parameters may include the voltage field applied to the treatment area, the pulse width, the frequency of pulsing, the number of pulses, or any combination thereof. In some embodiments, the electrical treatment parameters may be selected based at least in part on the conductive spacer in the electrode assembly. For example, an electrode assembly with a conductive spacer of the present disclosure may require less voltage to provide an effective treatment at a desired treatment depth throughout the treatment area than the voltage required to treat the treatment area with an electrode assembly having an insulative spacer or no spacer between the electrodes. This lower voltage may be possible at least in part because the conductive spacer allows a relatively high electric field to exist above the treatment area and extend into the treatment area more effectively. In some embodiments, the controller of the pulse generator system (such as the one shown by example in FIG. 1) may be configured to determine or recognize the selected electrode assembly and the conductivity of the conductive spacer and may also provide suggested electrical treatment parameter values for treatment. For example, when the electrode assembly is attached to the handle of the pulse generator system, the controller may receive information identifying the electrode assembly (e.g., a model number). The controller may access a memory that contains a database or table that includes information that can be used to determine electrical treatment parameter values for treatment of the given treatment area using the selected electrode assembly. The electrical treatment parameter values may be based on, for example, any combination of the conductivity of the conductive spacer, geometric cutout information of the conductive spacer, the conductivity of the treatment area, the size or shape of the treatment area, or the like.

In some embodiments, at optional step 1025, the user may apply the electrical treatment to the treatment area via the electrode assembly. Such treatment may include high-voltage or low-voltage pulses. The pulses may be nanosecond pulses, picosecond pulses, microsecond pulses, millisecond pulses, or the like. The electrical treatment may be, in some embodiments, Direct Current (“DC”) pulsing. In some embodiments, the electrical treatment may be Radio Frequency (“RF”) therapy.

FIG. 10B illustrates an example of another method 1050 for selecting electrode assembly and/or selecting parameters for electrical treatment. The method 1050 may be performed by a human user (e.g., a medical professional) operating the treatment device or system, for example, such as system 100 of FIG. 1. In some embodiments, the method 1050 may be performed by a controller of the treatment system or device. The method 1050 may include at step 1055, selecting an electrode assembly based at least in part on a conductive spacer of the electrode assembly. Step 1055 is described in detail above as optional step 1005 in process 1000 of FIG. 10A.

In some embodiments, at optional step 1060 a conductive gel may be applied on the surface of the treatment area to encourage contact between the electrode assembly electrodes and the treatment area and/or between the conductive spacer of the electrode assembly and the treatment area. Step 1060 is described in detail above as optional step 1010 in process 1000 of FIG. 10A.

At step 1065, the electrical treatment parameters may be selected for the electrical treatment to apply to the treatment area. Step 1065 is described in detail above as step 1020 in process 1000 of FIG. 10A.

FIG. 11 illustrates an example of another method 1100 for configuring a pulse generator system for electrical treatment or a method of operation of an electrical therapy device. The method 1100 may be performed by a controller of the pulse generator system. The controller may perform the method 1100 by having instructions stored within a memory of the controller that, when executed by one or more processors of the controller, cause the controller to perform the steps. At step 1105, the controller may determine or receive an indication of the exchangeable electrode assembly of the treatment applicator. For example, a human user may select an exchangeable electrode assembly and couple the exchangeable electrode assembly to a handle of the treatment applicator. In some implementations, the system may determine and suggest through the user interface which electrode assembly to use for a particular treatment area or for a particular treatment, for example, based on the information stored in the memory of the controller/processor of the system. The coupling may be mechanical, electrical, or both. The electrode assembly may include information that is transmitted upon coupling to the controller to indicate the type of electrode assembly including the size of the electrode assembly (e.g., 5 mm×5 mm, 10 mm×5 mm, 5 mm×10 mm, or the like), the conductivity of the conductive spacer, the number of electrodes, the type of the electrode assembly (e.g., whether it is a surface electrode, needle electrode, or combination electrode assembly), and/or the like. In some embodiments, a model number of the electrode assembly may be transmitted to the controller, and the controller may use the model number to identify the information about the electrode assembly in a table in memory, for example.

At step 1110, the controller determines the characteristics of the conductive spacer based on the indication of the exchangeable electrode assembly. For example, the indication may include the conductivity information (e.g., conductivity, conductive zones, or the like), or the information provided may allow the controller to look up the conductivity information of the conductive spacer. As another example, the indication may include the shape or other characteristics of the recess of the conductive spacer.

At step 1115, the controller determines suitable treatment configuration information, including for example electrical treatment parameters, based on the exchangeable electrode assembly. For example, the controller may look up configuration information in a table or database. The configuration information may include, for example, a preferred type of tissue to be treated with the electrode assembly, a peak voltage value for the treatment, a treatment duration for performing the electrical treatment, the treatment depth that the treatment is desired to reach, the minimum effective treatment value for the tissue that is being treated at the treatment depth, and/or the like.

In some embodiments, optional step 1120 is performed by the controller. The controller may use the configuration information based on the exchangeable electrode assembly selected to set configurable electrical treatment parameters for the electrical treatment. For example, the controller may set the peak voltage for the treatment, the voltage field to be applied, the pulse duration, the pulse frequency, the number of pulses, the pulse width, or the like. In some embodiments, optional step 1120 may be performed by a user.

At step 1125, in some embodiments the controller may optionally cause displaying the suitable treatment configuration information and/or the configurable electrical treatment parameters on a display device (e.g., interface 104). In some implementations, instead of displaying the suitable treatment configuration information and/or the treatment parameters, such information may be communicated or transmitted, for example, verbally (e.g., by sound), or by text. In some embodiments, the user may adjust the configurable parameters and/or modify the preferred or suitable treatment configuration information. For example, the user may modify the type of tissue being treated or the peak voltage to be used. Such modification may, in some embodiments, cause the controller to modify/update the determined suitable treatment configuration information based on, for example, the table or historical data. In some embodiments, information in memory of the controller may include protections that do not allow the configuration settings for a given exchangeable electrode assembly to provide unallowable treatments, for example, by limiting peak voltages or the like.

FIG. 12A illustrates a model 1200 of a treatment area 1215 for a surface electrode assembly having an insulated spacer 1210 between the electrodes 1205a, 1205b and surrounded by insulator 1220. The described surface electrodes 1205 having an insulated spacer 1210 may have been used in previous known treatments and do not implement the conductive spacer disclosed herein. This model 1200 is provided for the purpose of depicting the treatment area 1215 using an insulative spacer 1210 to show the substantial benefit of using the conductive spacer of the present disclosure as shown in FIG. 12B.

The model 1200 is based on a treatment area 1215 being treated with an insulative spacer 1210 between two surface electrodes 1205a and 1205b. The insulative spacer 1210 has a height that is 10% of its width. A voltage pulse is applied that may have, for example, 2 kV to 15 kV peak voltage. As shown using numerical values in the model, there are iso lines numbered from 1 to 10 that indicate a relative treatment value at each iso line. As shown, the treatment area 1215 directly below the center of the insulative spacer 1210 has a treatment value at the 2.0 iso line to a depth of approximately 2.5. However, the 4.0 iso line does not extend below the center of the insulative spacer 1210. For the purposes of discussion, we will assume the treatment area 1215 has an effective treatment value identified based on the 4.0 iso line. Directly under the electrodes 1205, the treatment value at the 4.0 iso line goes to a depth of approximately 2.25. As shown using an insulative spacer, the treatment is not distributed evenly throughout the treatment area 1215. The iso line associated with the effective treatment value for the treatment area may be as horizontal as possible to indicate an evenly distributed treatment to a consistent depth in the treatment area. In the case of model 1200, none of the iso lines indicate an evenly distributed treatment.

FIG. 12B illustrates a model 1250 of a treatment area 1265 for a surface electrode assembly having conductive spacer 1260 between the electrodes 1255a, 1255b and surrounded by insulator 1270. The model 1250 is based on a treatment area 1265 being treated with a conductive spacer 1260 between two surface electrodes 1255. The conductive spacer 1260 has a height that is approximately 10% of its width. The conductive spacer 1260 has a conductivity that is substantially the same as the conductivity of the treatment area 1265. A voltage pulse is applied that may have, for example, 2 kV to 15 kV peak voltage. The approximate treatment values at various depths are shown by the iso lines numbered as described in FIG. 12A. In model 1250, directly under the electrodes 1255, the treatment value at the 4.0 iso line goes to a depth of approximately 2.75, and the treatment value at the 4.0 iso line directly under the center of the conductive spacer 1260 goes to a depth of approximately 1.25. Note that the treatment is distributed substantially more evenly throughout the treatment area 1265 than through treatment area 1215, as depicted in FIG. 12A, when an insulative spacer 1210 is used. In this example, effective treatment to a depth of at least 1.25 is achieved throughout the treatment area 1265.

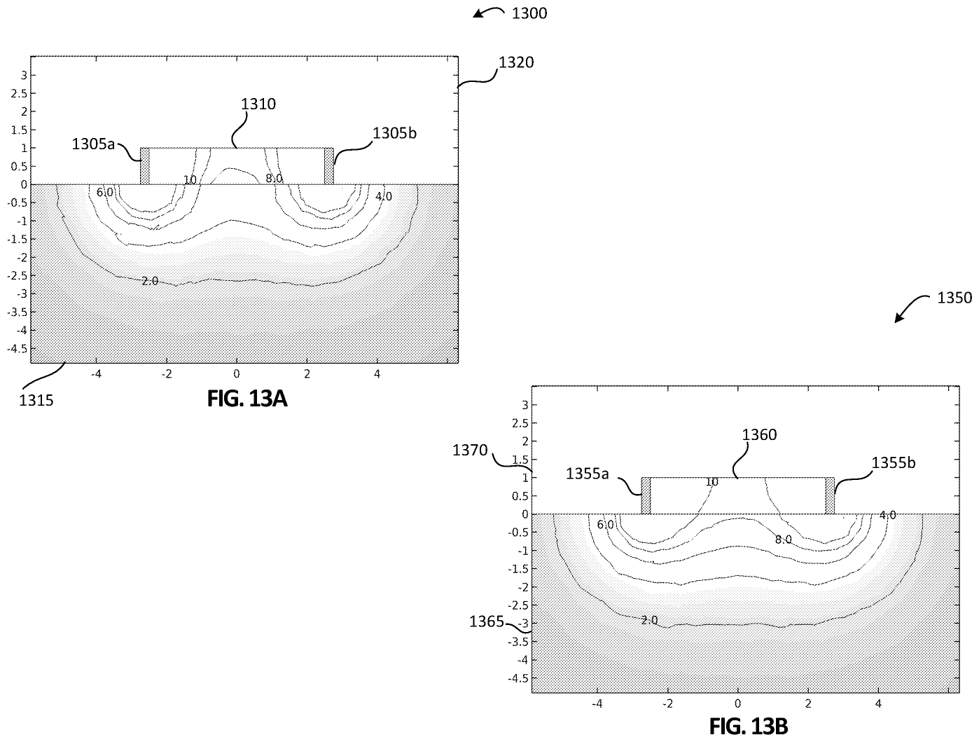

FIG. 13A illustrates a model 1300 of a treatment area 1315 for a surface electrode assembly having an insulated spacer 1310 between the electrodes 1305 and surrounded by insulator 1320. The surface electrode assembly depicted in FIG. 13A is a smaller size than the surface electrode assembly depicted in FIG. 12A. The distance between the electrodes 1305 is half the distance as between the electrodes 1205 in FIG. 12A. The described surface electrodes 1305 having an insulated spacer 1310 may have been used in previous treatments and does not implement the conductive spacer disclosed herein. This model 1300 is provided for the purpose of depicting the treatment area 1315 using an insulative spacer 1310 to show the substantial benefit of using the conductive spacer as shown in FIG. 13B.

The model 1300 is based on a treatment area 1315 being treated with an insulative spacer 1310 between two surface electrodes 1305a and 1305b. The insulative spacer 1310 has a height that is 20% of its width. A voltage pulse is applied that may have, for example, 2 kV to 15 kV peak voltage. The approximate treatment values at various depths are shown by the iso lines numbered as described in FIG. 12A. The treatment area 1315 directly below the insulative spacer 1310 has a treatment value at the 4.0 iso line to a depth of approximately 1. Directly under the electrodes 1305, the treatment value at the 4.0 iso line goes to a depth of approximately 1.75. However, the effective treatment value is not distributed evenly throughout the treatment area 1315 as is preferred.

FIG. 13B illustrates a model 1350 of a treatment area 1365 for a surface electrode assembly having conductive spacer 1360 between the electrodes 1355a, 1355b and surrounded by insulator 1370.

The model 1350 is based on a treatment area 1365 being treated with a conductive spacer 1360 between two surface electrodes 1355. The conductive spacer 1360 has a height that is approximately 20% of its width. The conductive spacer 1360 has a conductivity that is substantially the same as the conductivity of the treatment area 1365. A voltage pulse is applied that may have, for example, 2 kV to 15 kV peak voltage. The approximate treatment values at various depths are shown by the iso lines numbered as described in FIG. 12A. In model 1350, directly below the center of the conductive spacer 1360 the treatment value at the 4.0 iso line goes to a depth of approximately 1.6, and the treatment value at the 4.0 iso line directly under the electrodes 1355 goes to a depth of approximately 1.8. Note that the effective treatment value is distributed substantially more evenly throughout the treatment area 1365 than through treatment area 1315 when an insulative spacer 1310 is used. In this example, effective treatment to a depth of approximately 1.6 is achieved throughout the treatment area 1365.

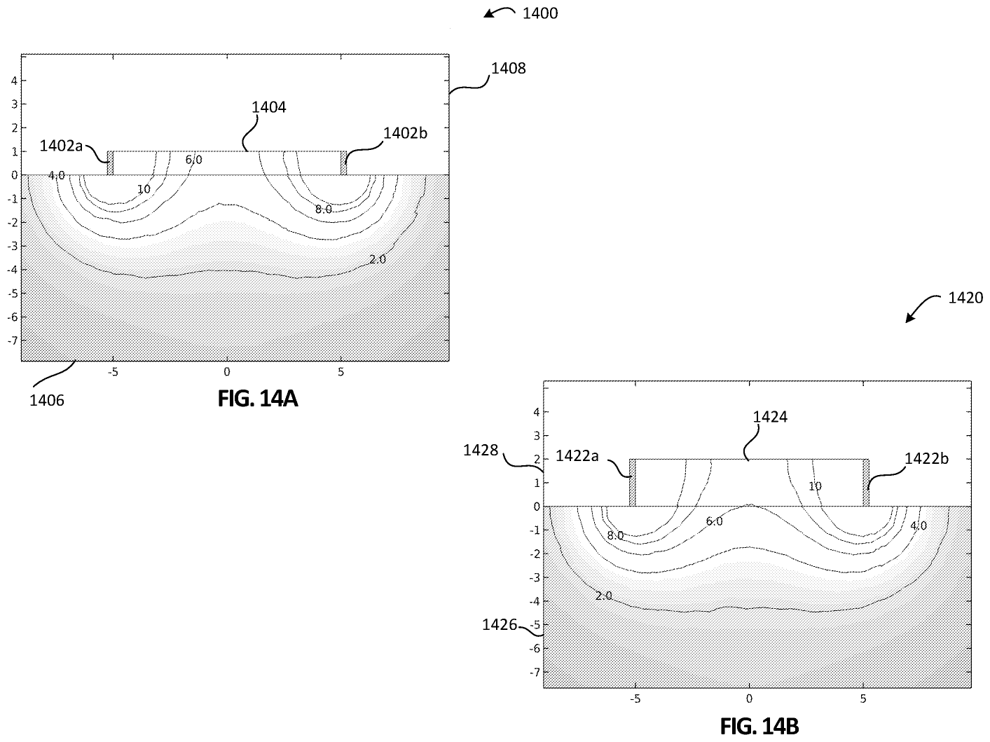

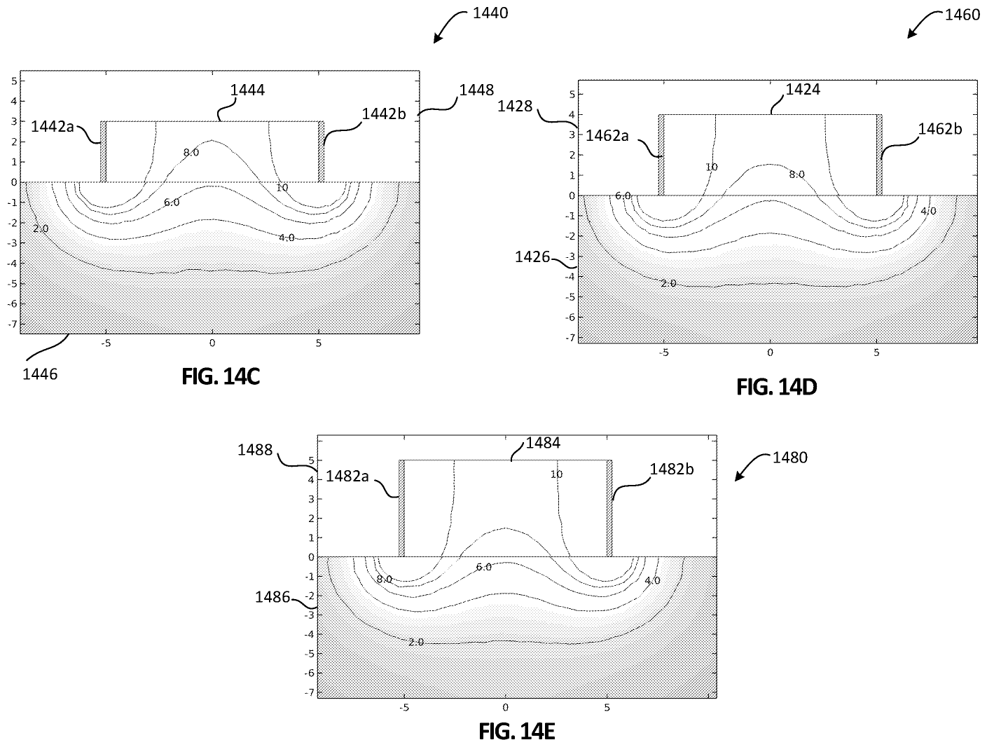

FIGS. 14A-14E can be used to analyze the benefit of increasing the height of the conductive spacer.

FIG. 14A illustrates a model 1400 of a treatment area 1406 for a surface electrode assembly having conductive spacer 1404 between the electrodes 1402a, 1402b. The area 1408 is insulative, such as an insulative housing.

The model 1400 is based on a treatment area 1406 being treated with a conductive spacer 1404 between two surface electrodes 1402. The conductive spacer 1404 has a height that is approximately 10% of its width. The conductive spacer 1404 has a conductivity that is substantially the same as the conductivity of the treatment area 1406. A voltage pulse is applied that may have, for example, 2 kV to 15 kV peak voltage. The approximate treatment values at various depths are shown by the iso lines numbered as described in FIG. 12A. The treatment area 1406 directly below the conductive spacer 1404 has a treatment value at the 4.0 iso line to a depth of approximately 1.25. Directly under the electrodes 1402, the treatment value at the 4.0 iso line goes to a depth of approximately 2.75.

FIG. 14B illustrates a model 1420 of a treatment area 1426 for a surface electrode assembly having conductive spacer 1424 between the electrodes 1422a, 1422b. The area 1428 is insulative, such as an insulative housing.

The model 1420 is based on a treatment area 1426 being treated with a conductive spacer 1424 between two surface electrodes 1422. The conductive spacer 1424 has a height that is approximately 20% of its width. The conductive spacer 1424 has a conductivity that is substantially the same as the conductivity of the treatment area 1426. A voltage pulse is applied that may have, for example, 2 kV to 15 kV peak voltage. The approximate treatment values at various depths are shown by the iso lines numbered as described in FIG. 12A. The treatment area 1426 directly below the conductive spacer 1424 has a treatment value at the 4.0 iso line to a depth of approximately 1.75. Directly under the electrodes 1422, the treatment value at the 4.0 iso line goes to a depth of approximately 2.75.

FIG. 14C illustrates a model 1440 of a treatment area 1446 for a surface electrode assembly having conductive spacer 1444 between the electrodes 1442a, 1442b. The area 1448 is insulative, such as an insulative housing.

The model 1440 is based on a treatment area 1446 being treated with a conductive spacer 1444 between two surface electrodes 1442. The conductive spacer 1444 has a height that is approximately 30% of its width. The conductive spacer 1444 has a conductivity that is substantially the same as the conductivity of the treatment area 1446. A voltage pulse is applied that may have, for example, 2 kV to 15 kV peak voltage. The approximate treatment values at various depths are shown by the iso lines numbered as described in FIG. 12A. The treatment area 1446 directly below the conductive spacer 1444 has a treatment value at the 4.0 iso line to a depth of approximately 1.75. Directly under the electrodes 1442, the treatment value at the 4.0 iso line goes to a depth of approximately 2.75.

FIG. 14D illustrates a model 1460 of a treatment area 1466 for a surface electrode assembly having conductive spacer 1464 between the electrodes 1462a, 1462b. The area 1468 is insulative, such as an insulative housing.

The model 1460 is based on a treatment area 1466 being treated with a conductive spacer 1464 between two surface electrodes 1462. The conductive spacer 1464 has a height that is approximately 40% of its width. The conductive spacer 1464 has a conductivity that is substantially the same as the conductivity of the treatment area 1466. A voltage pulse is applied that may have, for example, 2 kV to 15 kV peak voltage. The approximate treatment values at various depths are shown by the iso lines numbered as described in FIG. 12A. The treatment area 1466 directly below the conductive spacer 1464 has a treatment value at the 4.0 iso line to a depth of approximately 2. Directly under the electrodes 1462, the treatment value at the 4.0 iso line goes to a depth of approximately 2.75.

FIG. 14E illustrates a model 1480 of a treatment area 1486 for a surface electrode assembly having conductive spacer 1484 between the electrodes 1482a, 1482b. The area 1488 is insulative, such as an insulative housing.

The model 1480 is based on a treatment area 1486 being treated with a conductive spacer 1484 between two surface electrodes 1482. The conductive spacer 1484 has a height that is approximately 50% of its width. The conductive spacer 1484 has a conductivity that is substantially the same as the conductivity of the treatment area 1486. A voltage pulse is applied that may have, for example, 2 kV to 15 kV peak voltage. The approximate treatment values at various depths are shown by the iso lines numbered as described in FIG. 12A. The treatment area 1486 directly below the conductive spacer 1484 has a treatment value at the 4.0 iso line to a depth of approximately 2. Directly under the electrodes 1482, the treatment value at the 4.0 iso line goes to a depth of approximately 2.75.

As shown by FIGS. 14A-14E, increasing the height to width ratio of the conductive spacer increases the depth of effective treatment value in the treatment area under the conductive spacer, but only to a point. Other models using various other tissues and conductivities of the conductive spacer as well as different size electrode assemblies (e.g., 5 mm×5 mm, 5 mm×10 mm) indicate there is an increase in effective treatment depth when the height of the conductive spacer is approximately between twenty percent and sixty percent (20%-60%) of the width of the conductive spacer over using a conductive spacer with a smaller height to width ratio. The increase in the effective treatment depth tapers off beyond 70%. A conductive spacer having a height of approximately forty percent (40%) of the width of the conductive spacer provides good results. Accordingly, in certain embodiments, a conductive spacer having a height of 4 mm and a width of 10 mm (e.g., in a 5 mm×10 mm electrode assembly) provides increased treatment depth and uniformity over a conductive spacer having a height of less than 4 mm and a width of 10 mm. Similarly, a conductive spacer having a height of 2 mm and a width of 5 mm (e.g., in a 5 mm×5 mm electrode assembly) provides increased treatment depth over a conductive spacer having a height of less than 2 mm and a width of 5 mm.

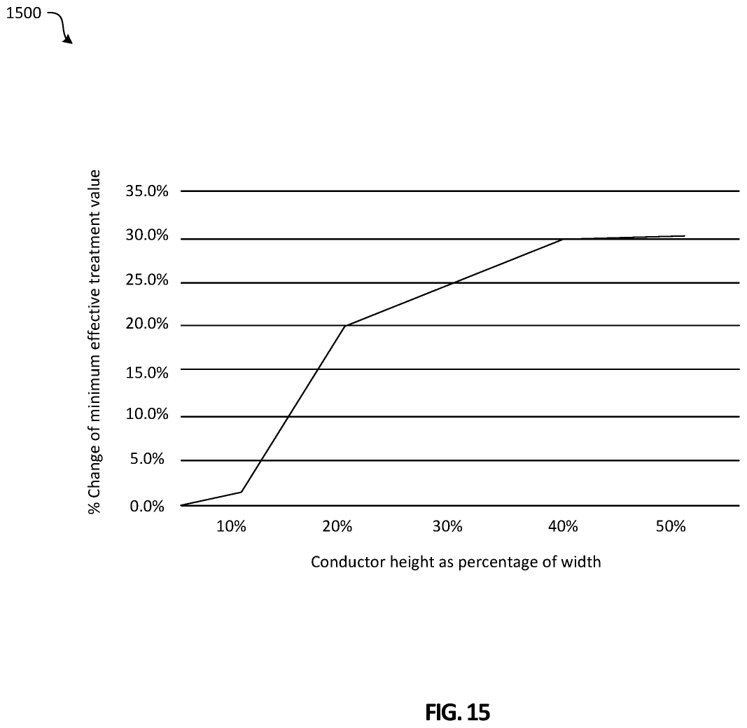

FIG. 15 illustrates an example of a graph 1500 of a percentage change of minimum effective treatment value as the height of the conductive spacer is increased as a percentage of its width based on the data collected in models 1400, 1420, 1440, 1460, and 1480. For example, the percent change of the effective electric field with a conductive spacer having a 10% height to width ratio to a conductive spacer having a 40% height to width ratio is approximately thirty percent (30%).

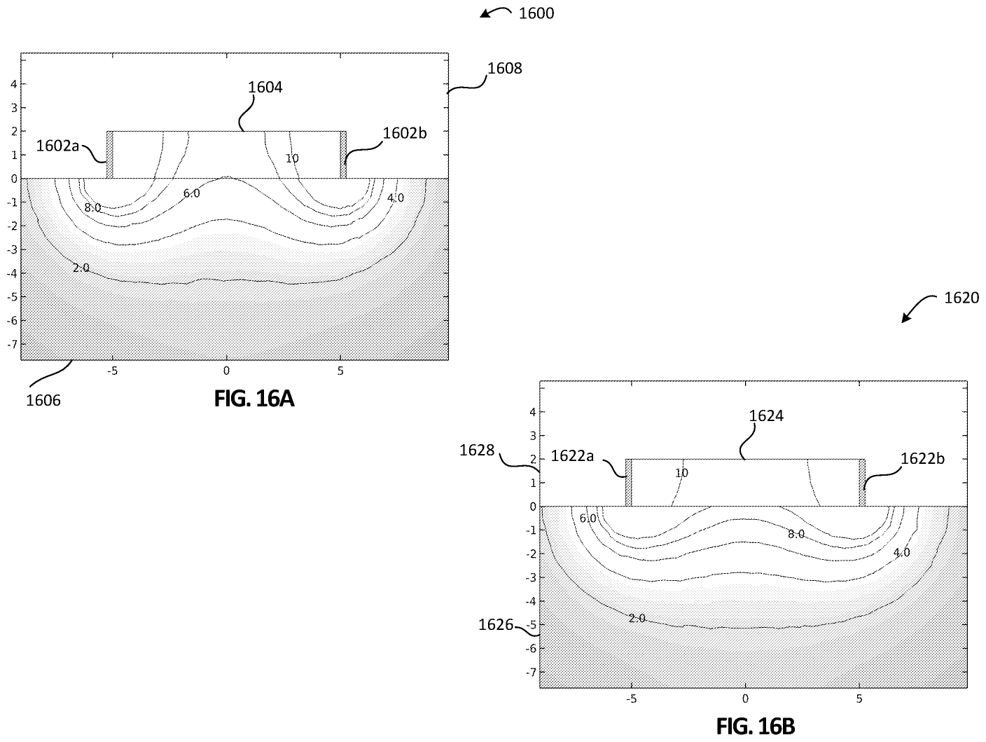

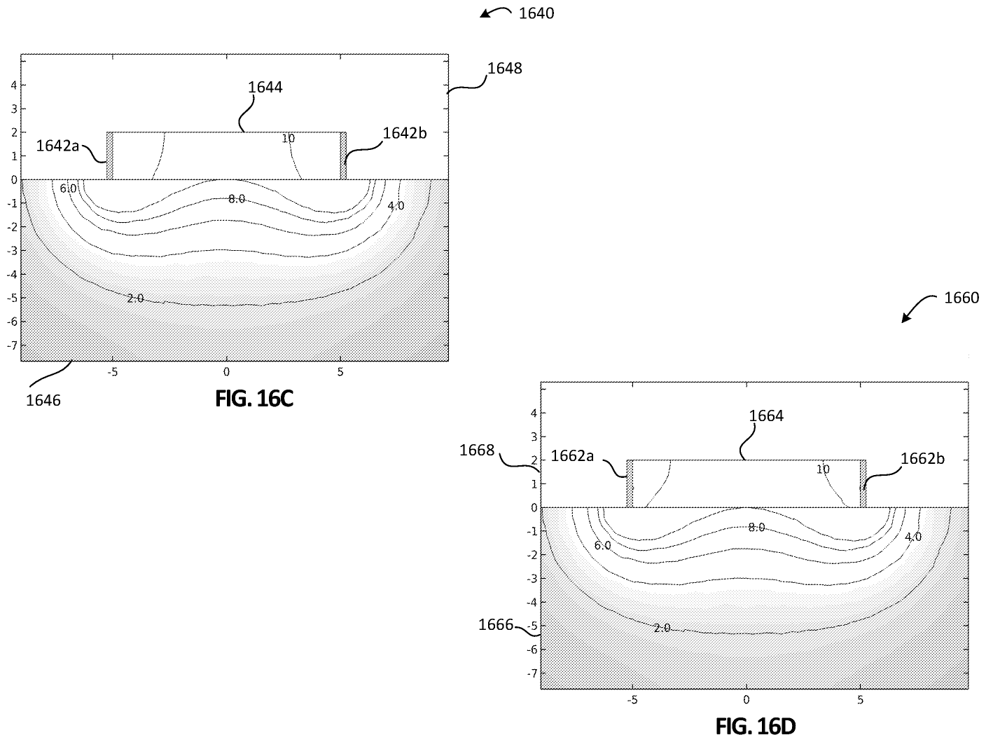

FIGS. 16A-16D can be used to analyze the benefit of increasing the conductivity of the conductive spacer.

FIG. 16A illustrates a model 1600 of a treatment area 1606 for a surface electrode assembly having conductive spacer 1604 between the electrodes 1602a, 1602b. The area 1608 is insulative, such as an insulative housing.

The model 1600 is based on a treatment area 1606 being treated with a conductive spacer 1604 between two surface electrodes 1602. The conductive spacer 1604 has a height that is approximately 20% of its width. The conductive spacer 1604 has a conductivity that is substantially the same as the conductivity of the treatment area 1606. A voltage pulse is applied that may have, for example, 2 kV to 15 kV peak voltage. The approximate treatment values at various depths are shown by the iso lines numbered as described in FIG. 12A. The treatment area 1606 directly below the conductive spacer 1604 has a treatment value at the 4.0 iso line to a depth of approximately 1.75. Directly under the electrodes 1602, the treatment value at the 4.0 iso line goes to a depth of approximately 2.75.

FIG. 16B illustrates a model 1620 of a treatment area 1626 for a surface electrode assembly having conductive spacer 1624 between the electrodes 1622a, 1622b. The area 1628 is insulative, such as an insulative housing.

The model 1620 is based on a treatment area 1626 being treated with a conductive spacer 1624 between two surface electrodes 1622. The conductive spacer 1624 has a height that is approximately 20% of its width. The conductive spacer 1624 has a conductivity that is approximately 10-times (10×) the conductivity of the treatment area 1626. A voltage pulse is applied that may have, for example, 2 kV to 15 kV peak voltage. The approximate treatment values at various depths are shown by the iso lines numbered as described in FIG. 12A. The treatment area 1626 directly below the conductive spacer 1624 has a treatment value at the 4.0 iso line to a depth of approximately 2.75. Directly under the electrodes 1622, the treatment value at the 4.0 iso line goes to a depth of approximately 3 mm Note that this is a substantial improvement over the conductive spacer 1604 with a conductivity substantially equal to the conductivity of the treatment area 1606.

FIG. 16C illustrates a model 1640 of a treatment area 1646 for a surface electrode assembly having conductive spacer 1644 between the electrodes 1642a, 1642b. The area 1648 is insulative, such as an insulative housing.

The model 1640 is based on a treatment area 1646 being treated with a conductive spacer 1644 between two surface electrodes 1642. The conductive spacer 1644 has a height that is approximately 20% of its width. The conductive spacer 1644 has a conductivity that is approximately 100-times (100×) the conductivity of the treatment area 1646. A voltage pulse is applied that may have, for example, 2 kV to 15 kV peak voltage. The approximate treatment values at various depths are shown by the iso lines numbered as described in FIG. 12A. The treatment area 1646 directly below the conductive spacer 1644 has a treatment value at the 4.0 iso line to a depth of approximately 3. Directly under the electrodes 1642, the treatment value at the 4.0 iso line goes to a depth of approximately 3.25. Note that this is even more improvement over the conductive spacer 1624 with conductivity 10× the conductivity of the treatment area 1646 because the effective treatment depth is increased.

FIG. 16D illustrates a model 1660 of a treatment area 1666 for a surface electrode assembly having conductive spacer 1664 between the electrodes 1662a, 1662b. The area 1668 is insulative, such as an insulative housing.

The model 1660 is based on a treatment area 1666 being treated with a conductive spacer 1664 between two surface electrodes 1662. The conductive spacer 1664 has a height that is approximately 20% of its width. The conductive spacer 1644 has a conductivity that is approximately 1000-times (1000×) the conductivity of the treatment area 1646. A voltage pulse is applied that may have, for example, 2 kV to 15 kV peak voltage. The approximate treatment values at various depths are shown by the iso lines numbered as described in FIG. 12A. The treatment area 1666 directly below the conductive spacer 1664 has a treatment value at the 4.0 iso line to a depth of approximately 3. Directly under the electrodes 1662, the treatment value at the 4.0 iso line goes to a depth of approximately 3.25. Note that there is not noticeable improvement over the conductive spacer 1644 with conductivity of 100× the treatment area 1646.

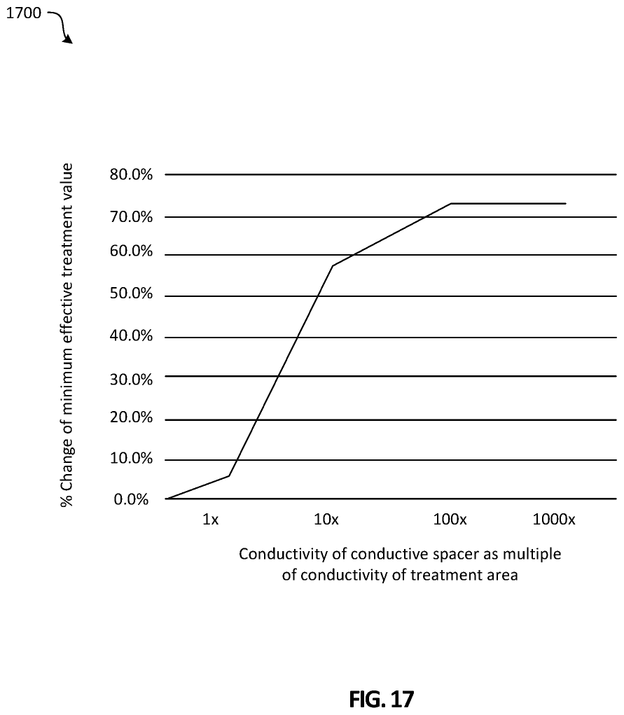

FIG. 17 illustrates a graph 1700 of a percentage change of minimum effective treatment values as the conductivity of the conductive spacer is increased as a multiple of the conductivity of the treatment area based on the data collected in models 1600, 1620, 1640, and 1660. For example, the percent change of the minimum effective treatment value between a conductive spacer having a conductivity of substantially the same conductivity of the treatment area and a conductive spacer having a conductivity of 100× the conductivity of the treatment area is approximately seventy-two percent (72%).

FIGS. 18A-18D illustrate alternative configurations and results for three-row penetrating and combination electrode assemblies. The areas 1808, 1828, 1848, 1868 are insulative, such as an insulative housing.

FIG. 18A illustrates a model 1800 for a three-row needle electrode assembly with insulated spacers 1804a, 1804b between the needle electrodes 1802a, 1802b, and 1802c.

The model 1800 of FIG. 18A is based on a treatment area 1806 being treated with a three-row needle electrode configuration. A voltage pulse is applied that may have, for example, 2 kV to 15 kV peak voltage. The approximate treatment values at various depths are shown by the iso lines numbered as described in FIG. 12A. The treatment area 1806 directly below the needle electrodes 1802a and 1802c has a treatment value at the 4.0 iso line to a depth of approximately 4. Directly below the needle electrode 1802b has a treatment value at the 4.0 iso line to a depth of approximately 5.25. Directly under the space between the needle electrodes 1802, the treatment value at the 4.0 iso line goes to a depth of between approximately 4 and 5.25. The middle row of needle electrodes 1802b effectively ensures that the area between the edge electrodes 1802a and 1802c receives treatment, but it is at a substantial cost. First, the treatment depth is even greater at the center of the electrode assembly. Second, the additional row of needles adds to the “bed of nails” phenomenon, making penetration difficult. Third, the manufacturing cost and complexity of a three-row needle electrode assembly is substantially more than a two-row needle electrode assembly.

FIG. 18B illustrates a model 1820 for a three-row needle electrode assembly with conductive spacers 1824a, 1824b between the needle electrodes 1822a, 1822b, and 1822c.

The model 1820 of FIG. 18B is based on a treatment area 1826 being treated with conductive spacers 1824a and 1824b between three needle electrodes 1822a, 1822b, 1822c. The conductive spacers 1824 have a conductivity that is substantially the same as the conductivity of the treatment area 1826. The conductive spacers 1824a, 1824b, each have a height that is approximately 80% of its width and 40% of the total width between electrode 1822a and 1822c. A voltage pulse is applied that may have, for example, 2 kV to 15 kV peak voltage. The approximate treatment values at various depths are shown by the iso lines numbered as described in FIG. 12A. The treatment area 1826 directly below the needle electrodes 1822a and 1822c has a treatment value at the 4.0 iso line to a depth of approximately 4. Directly below the needle electrode 1822b has a treatment value at the 4.0 iso line to a depth of approximately 5.25. Directly under the space between the needle electrodes 1822, the treatment value at the 4.0 iso line goes to a depth of between approximately 4 and 5.25. The middle row of needle electrodes 1822b effectively ensures that the area between the edge electrodes 1822a and 1822c receives treatment, but the conductive spacer at this conductivity appears to have little impact over a three-row needle electrode without a conductive spacer.

FIG. 18C illustrates a model 1840 for a combination electrode assembly with needle electrodes 1842a and 1842c and a surface electrode 1842b with an insulative spacer 1844a, 1844b between the needle electrodes 1842a, 1842c and the surface electrode 1842b.

The model 1840 of FIG. 18C is based on a treatment area 1846 being treated with two exterior rows of needle electrodes 1842a, 1842c and a center surface electrode 1842b positioned between the two rows of needle electrodes 1842a, 1842c in place of a third row of needle electrodes. A voltage pulse is applied that may have, for example, 2 kV to 15 kV peak voltage. The approximate treatment values at various depths are shown by the iso lines numbered as described in FIG. 12A. The treatment area 1846 directly below the needle electrodes 1842a and 1842c has a treatment value at the 4.0 iso line to a depth of approximately 2.75. Directly below the surface electrode 1842b has a treatment value to a depth of approximately 3.75. Directly under the space between the needle electrodes 1842a, 1842c and the surface electrode 1842b, the treatment value goes to a depth of between 2.75 and 3.75. The middle surface electrode 1842b assists in distributing the treatment throughout the area between the edge electrodes 1842a and 1842c, but the distribution is not as even as it was with three rows of needle electrodes.

FIG. 18D illustrates a model 1860 for a combination electrode assembly with a conductive spacer 1864a between needle electrode 1862a and surface electrode 1862b and conductive spacer 1864b between needle electrode 1862c and surface electrode 1862b.

The model 1860 of FIG. 18D is based on a treatment area 1866 being treated with two exterior rows of needle electrodes 1862a, 1862c and a center surface electrode 1862b positioned between the two rows of needle electrodes 1862a, 1862c in place of a third row of needle electrodes. The conductive spacers 1864 have a conductivity that is substantially the same as the conductivity of the treatment area 1866. The conductive spacers 1824a, 1824b, each have a height that is approximately 80% of its width and 40% of the total width between electrode 1862a and 1862c. A voltage pulse is applied that may have, for example, 2 kV to 15 kV peak voltage. The approximate treatment values at various depths are shown by the iso lines numbered as described in FIG. 12A. The treatment area 1866 directly below the needle electrodes 1862a and 1862c has a treatment value at the 4.0 iso line to a depth of approximately 3.5. Directly below the surface electrode 1862b has an effective treatment value to a depth of approximately 4.25. Directly under the space between the needle electrodes 1862a, 1862c and the surface electrode 1862b, the effective treatment value goes to a depth of between 3.5 and 4.25. This configuration provides a substantial improvement of distribution of effective treatment value throughout the treatment area 1866 while providing the improvements known from eliminating a row of needle electrodes (e.g., avoiding the “bed of nails” phenomenon) and lowering manufacturing cost and complexity by replacing a row of needle electrodes with a single surface electrode.

FIGS. 19A-19C illustrate alternative configurations and results for electrode assemblies with surface electrodes and a conductive spacer with multiple conductivity zones. The areas 1908, 1928, and 1948 are insulative, such as an insulative housing.

FIG. 19A illustrates a model 1900 for an electrode assembly having surface electrodes 1902a, 1902b with a conductive spacer having a first conductive zone 1904a, 1904b and a second conductive zone 1910 between the surface electrodes 1902.

The model 1900 of FIG. 19A is based on a treatment area 1906 being treated with a surface electrode assembly having a conductive spacer with conductive zones 1904, 1910 between surface electrodes 1902. The conductivity of the first zone 1904 may be 10× the conductivity of the treatment area 1906, and the conductivity of the second zone 1910 may be substantially the same as the conductivity of the treatment area 1906. The two conductivity zones shown 1904, 1910 are sharply delineated in this example, but in some embodiments a gradual change of the conductivity in the conductive spacer may be used. The conductive spacer has a height that is approximately 20% of its width. A voltage pulse is applied that may have, for example, 2 kV to 15 kV peak voltage. The approximate treatment values at various depths are shown by the iso lines numbered as described in FIG. 12A. The treatment area 1906 directly below the surface electrodes 1902a and 1902b has a treatment value at the 4.0 iso line to a depth of approximately 3.25. Directly under the conductive zones 1904, 1910, the effective treatment value goes to a depth of approximately 4 mm and is substantially evenly distributed. Referring back to FIG. 16B, the conductive spacer 1624 has a height that is approximately 20% of its width and a conductivity that is approximately 10× the conductivity of the treatment area 1626. In comparing the distribution of the treatment shown in model 1620 to that in model 1900, the distribution is substantially more even in the model 1900 where conductive zones in the conductive spacer are used.

FIG. 19B illustrates a model 1920 for an electrode assembly with surface electrodes 1922a, 1922b with a conductive spacer having two conductivity zones 1924a, 1924b, and 1930 between the surface electrodes 1922.

The model 1920 of FIG. 19B is based on a treatment area 1926 being treated with a surface electrode assembly having a conductive spacer with conductive zones 1924, 1930 between surface electrodes 1922. The conductivity of the first zone 1924 may be 10× the conductivity of the treatment area 1926, and the conductivity of the second zone 1930 may be substantially zero, or insulative. The two conductivity zones shown 1924, 1930 are sharply delineated in this example, but in some embodiments a gradual change of the conductivity in the conductive spacer may be used. The conductive spacer has a height that is approximately 20% of its width. A voltage pulse is applied that may have, for example, 2 kV to 15 kV peak voltage. The approximate treatment values at various depths are shown by the iso lines numbered as described in FIG. 12A. The treatment area 1926 directly below the surface electrodes 1922a and 1922b has a treatment value at the 4.0 iso line to a depth of approximately 3. Directly under the conductive zones 1924, 1930, the treatment value goes to a depth of approximately 3.75. The area under the conductive zones 1924, 1930 is fairly evenly distributed, though the area shown in FIG. 19A under conductive zones 1904, 1910 appears slightly more evenly distributed.

FIG. 19C illustrates a model 1940 for an electrode assembly with surface electrodes 1942a, 1942b with a conductive spacer having two conductive zones 1944a, 1944b, and 1950 between the surface electrodes 1942.

The model 1940 of FIG. 19C is based on a treatment area 1946 being treated with a surface electrode assembly having a conductive spacer with conductive zones 1944, 1950 between surface electrodes 1942. The conductivity of the first zone 1924 may be 100× the conductivity of the treatment area 1926, and the conductivity of the second zone 1930 may be 10× the conductivity of the treatment area 1946. The two conductive zones shown 1944, 1950 are sharply delineated in this example, but in some embodiments a gradual change of the conductivity in the conductive spacer may be used. The conductive spacer has a height that is approximately 20% of its width. A voltage pulse is applied that may have, for example, 2 kV to 15 kV peak voltage. The approximate treatment values at various depths are shown by the iso lines numbered as described in FIG. 12A. The treatment area 1946 directly below the surface electrodes 1942a and 1942b has a treatment value at the 4.0 iso line to a depth of approximately 3.25. Directly under the conductive zones 1944, 1950, the treatment value at the 4.0 iso line goes to a depth of approximately 3.75. The area under the conductive zones 1944, 1950 is fairly evenly distributed, however, the area shown in FIG. 19A under conductive zones 1904, 1910 appears equally evenly distributed and may have a smaller cost due to the lower cost of the lower conductivity of the conductive spacer.

FIGS. 20A and 20B illustrate alternative configurations and results for a three-row combination electrode assembly. The areas 2008 and 2028 are insulative, such as an insulative housing.

FIG. 20A illustrates a model 2000 for an electrode assembly having three rows of surface electrodes 2002a, 2002b, 2002c with a conductive spacer having a first conductive zone 2004a, 2004b, 2004c, 2004d and a second conductive zone 2010a, 2010b between the surface electrodes 2002.

The model 2000 of FIG. 20A is based on a treatment area 2006 being treated with a three-row surface electrode assembly having a conductive spacer with conductive zones 2004, 2010 between surface electrodes 2002. The conductivity of the first zone 2004 may be 10× the conductivity of the treatment area 2006, and the conductivity of the second zone 2010 may be substantially zero, or insulative. The two conductive zones shown 2004, 2010 are sharply delineated in this example, but in some embodiments a gradual change of the conductivity in the conductive spacer may be used. The conductive spacer has a height that is approximately 20% of the total width (i.e., the distance between the electrodes 2002a and 2002c). The width of each conductive zone 2004 is 40% of the distance between the electrodes the conductive spacer is disposed between. In other words, the conductive spacer 2004a is 40% of the distance between the electrodes 2002a and 2002b. A voltage pulse is applied that may have, for example, 1 kV to 15 kV peak voltage. The approximate treatment values at various depths are shown by the iso lines numbered as described in FIG. 12A. The treatment area 2006 directly below the surface electrodes 2002a and 2002c has a treatment value at the 4.0 iso line to a depth of approximately 3. Directly below the surface electrode 2002b has a treatment value at the 4.0 iso line to a depth of approximately 4.25.

FIG. 20B illustrates a model 2020 for a three-row electrode assembly having two exterior rows of needle electrodes 2022a, 2022b and center surface electrode 2032 and having conductive spacers with a first conductive zone 2024a, 2024b and a second conductive zone 2030a, 2030b.

The model 2020 of FIG. 20B is based on a treatment area 2026 being treated with a conductive spacer having two conductive zones 2030a, 2024a between needle electrode 2022a and surface electrode 2032 and a conductive spacer having two conductive zones 2030b, 2024b between needle electrodes 2022b and surface electrode 2032. The conductivity of the first zone 2030 may be substantially zero, or insulative, and the conductivity of the second zone 2024 may be approximately 10× the conductivity of the treatment area 2026. The two conductive zones shown 2024, 2030 are sharply delineated in this example, but in some embodiments a gradual change of the conductivity in the conductive spacer may be used. The conductive spacer has a height that is approximately 20% of the total width (i.e., the distance between the electrodes 2022a and 2022b). The width of each conductive zone 2024 is 40% of the distance between the electrodes the conductive spacer is disposed between. In other words, the conductive spacer 2024a is 40% of the distance between the electrodes 2022a and 2032. A voltage pulse is applied that may have, for example, 2 kV to 15 kV peak voltage. The approximate treatment values at various depths are shown by the iso lines numbered as described in FIG. 12A. The treatment area 2026 directly below the needle electrodes 2022a and 2022b has a treatment value at the 4.0 iso line to a depth of approximately 4. Directly below the surface electrode 2032 has a treatment value at the 4.0 iso line to a depth of approximately 4.75. The treatment is evenly distributed with this configuration, but the penetrating electrodes carry the same issues previously described, so a surface electrode configuration such as that shown in FIGS. 19A, 19B, 19C, and/or 20A may be preferred.

FIGS. 21A through 21E illustrate alternative configurations and results for a surface electrode assembly having conductive spacers with recesses. The varying recesses, for example, geometric cutouts may be similar to the conductive spacers having conductive zones as shown and discussed in FIGS. 19A-20B. The areas 2108, 2128, 2148, 2168, and 2188 are insulative, such as an insulative housing.

FIG. 21A illustrates a model 2100 of a treatment area 2106 for a surface electrode assembly having conductive spacer 2104 between the electrodes 2102a, 2102b and surrounded by insulator 2108.

The model 2100 is based on a treatment area 2106 being treated with a geometrically shaped conductive spacer 2104 between two surface electrodes 2102. The conductive spacer 2104 has a “V” shaped recess 2116. The conductive spacer 2104 has a first height at 2110 of 40% of the conductive spacer 2104 width and a second height at 2114 of 12.5% of the first height. The angle of the edge extending from the first height to the second height is 100 degrees. The conductive spacer 2104 has a conductivity that is substantially the same as the treatment area 2106. Notice that the recess 2116 operates effectively as an insulator. A voltage pulse is applied that may have, for example, 2 kV to 15 kV peak voltage. The approximate treatment values at various depths are shown by the iso lines numbered as described in FIG. 12A. In this embodiment, the treatment area 2106 directly below the center of the conductive spacer 2104 has a treatment value at the 4.0 iso line to a depth of approximately 4.25. Directly under the electrodes 2102, the treatment value at the 4.0 iso line goes to a depth of approximately 3.25. The distribution of the effective treatment is not as evenly distributed as that described in FIG. 19B, having a conductive spacer with conductive zones.

FIG. 21B illustrates a model 2120 of a treatment area 2126 for a surface electrode assembly having conductive spacer 2124 between the electrodes 2122a, 2122b. The area 2128 is insulative, such as an insulative housing.

The model 2120 is based on a treatment area 2126 being treated with a conductive spacer 2124 between two surface electrodes 2122. The conductive spacer 2124 has a “rounded V” shaped geometric cutout 2136. The conductive spacer 2124 has a first height at 2130 of 40% of the conductive spacer 2124 width and a second height at 2134 of 12.5% of the first height. In this example, the angle of the edge 2132 extending between the first height and the second height is 90 degrees. In some embodiments, the radius of curvature of the cutout in a “U” or “rounded V” shaped recess may help smooth the electric field. Accordingly, the radius of curvature may be adjusted to create a more uniform field throughout the treatment area in some embodiments. The conductive spacer 2124 is disposed between two surface electrodes 2122. The conductive spacer 2104 has a conductivity that is substantially the same as the treatment area 2106. Notice that the recess 2116 operates effectively as an insulator. A voltage pulse is applied that may have, for example, up to 15 kV peak voltage. The approximate treatment values at various depths are shown by the iso lines numbered as described in FIG. 12A. The treatment area 2126 directly below the conductive spacer 2124 has a treatment value at the 4.0 iso line to a depth of approximately 4.25. Directly under the electrodes 2122, the treatment value at the 4.0 iso line goes to a depth of approximately 3.25.

FIG. 21C illustrates a model 2140 of a treatment area 2146 for a surface electrode assembly having a conductive spacer 2144 between the electrodes 2142a, 2142b. The area 2148 is insulative, such as an insulative housing.

The model 2140 is based on a treatment area 2146 being treated with a conductive spacer 2144 between two surface electrodes 2142. The conductive spacer 2144 has a “U” shaped recess 2156. The conductive spacer 2144 has a first height at 2150 of 40% of the conductive spacer 2144 width and a second height at 2154 of 12.5% of the first height. The angle of the edge extending from the first height to the second height is 10 degrees. The conductive spacer 2144 has a conductivity that is substantially the same as the treatment area 2146. Notice that the recess 2156 operates effectively as an insulator. A voltage pulse is applied that may have, for example, 2 kV to 15 kV peak voltage. The approximate treatment values at various depths are shown by the iso lines numbered as described in FIG. 12A. The treatment area 2146 directly below the conductive spacer 2144 has a treatment value at the 4.0 iso line to a depth of approximately 4.25. Directly under the electrodes 2142, the treatment value at the 4.0 iso line goes to a depth of approximately 3.25.

FIG. 21D illustrates a model 2160 of a treatment area 2166 for a surface electrode assembly having a conductive spacer 2164 between the electrodes 2162a, 2162b. The area 2168 is insulative, such as an insulative housing.

The model 2160 is based on a treatment area 2166 being treated with a conductive spacer 2164 between two surface electrodes 2162. The conductive spacer 2164 has a “flat V” shaped recess 2176. The conductive spacer 2164 has a first height at 2170 of 40% of the conductive spacer 2164 width and a second height at 2174 of 12.5% of the first height. The angle of the edge extending from the first height to the second height is 100 degrees. The conductive spacer 2164 has a conductivity that is substantially the same as the treatment area 2166. Notice that the recess 2176 operates effectively as an insulator. A voltage pulse is applied that may have, for example, 5 kV to 15 kV peak voltage. The approximate treatment values at various depths are shown by the iso lines numbered as described in FIG. 12A. The treatment area 2166 directly below the conductive spacer 2164 has a treatment value at the 4.0 iso line to a depth of approximately 4.25. Directly under the electrodes 2162, the treatment value at the 4.0 iso line goes to a depth of approximately 3.25.

FIG. 21E illustrates a model 2180 of a treatment area 2186 for a surface electrode assembly having a conductive spacer 2184 between the electrodes 2182a, 2182b. The area 2188 is insulative, such as an insulative housing.

The model 2180 is based on a treatment area 2186 being treated with a conductive spacer 2184 between two surface electrodes 2182. The conductive spacer 2184 has a different “flat V” shaped recess 2196. The conductive spacer 2184 has a first height at 2190 of 40% of the conductive spacer 2184 width and a second height at 2194 of 12.5% of the first height. The angle of the edge extending from the first height to the second height is 10 degrees. The conductive spacer 2184 has a conductivity that is substantially the same as the treatment area 2186. Notice that the recess 2196 operates effectively as an insulator. A voltage pulse is applied that may have, for example, 5 kV to 15 kV peak voltage. The approximate treatment values at various depths in this example are shown by the iso lines numbered as described in FIG. 12A. The treatment area 2186 directly below the conductive spacer 2184 has a treatment value at the 4.0 iso line to a depth of approximately 4.5. Directly under the electrodes 2182, the treatment value at the 4.0 iso line goes to a depth of approximately 3.25.

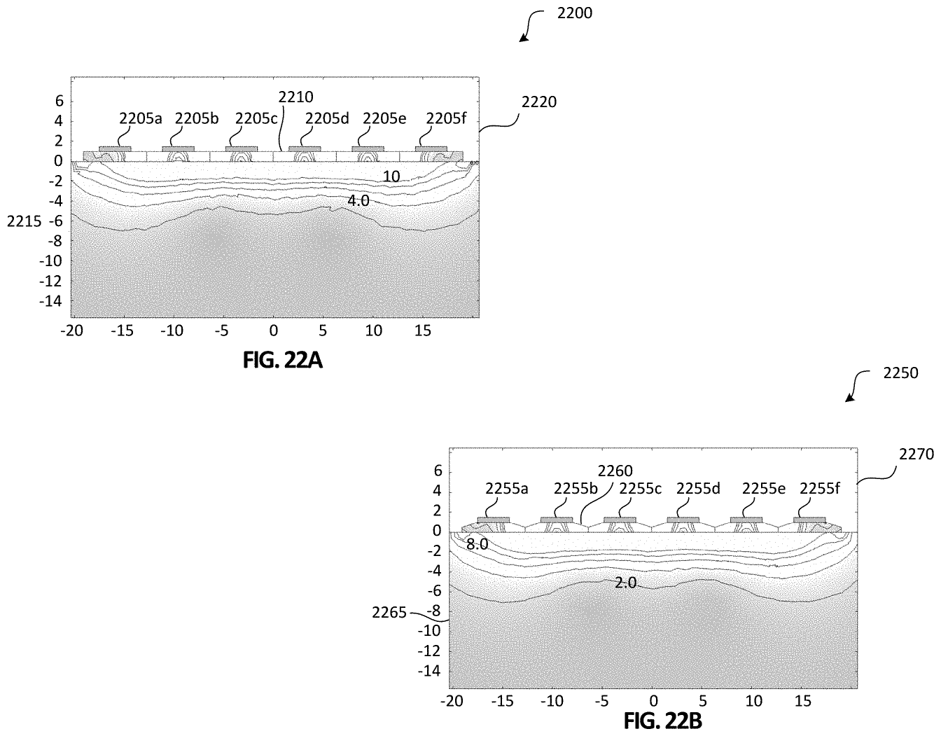

FIGS. 22A and 22B depict strip electrodes on a conductive spacer having differing geometries.

FIG. 22A illustrates a model 2200 of a treatment area 2215 for an electrode assembly having strip electrodes 2205a, 2205b, 2205c, 2205d, 2205e, and 2205f with a conductive spacer 2210 disposed upon the strip electrodes 2205. The conductive spacer 2210 may have a conductivity that is substantially the same as the conductivity of the treatment area 2215. A voltage pulse is applied that may have, for example, 2 kV to 20 kV peak voltage. The approximate treatment values at various depths are shown by the iso lines numbered as described in FIG. 12A. The treatment area 2215 has a treatment value at the 4.0 iso line to a depth ranging from 5.0 to 7.0.

FIG. 22B illustrates a model 2250 of a treatment area 2265 for an electrode assembly having strip electrodes 2255a, 2255b, 2255c, 2255d, 2255e, and 2255f with a conductive spacer 2260 disposed upon the strip electrodes 2255. The conductive spacer 2260 may have geometric or other recesses between the strip electrodes 2255. As depicted, the recesses may be “V” shaped. The conductive spacer 2260 may have a conductivity that is substantially the same as the conductivity of the treatment area 2265. A voltage pulse is applied that may have, for example, up to 20 kV peak voltage. The approximate treatment values at various depths are shown by the iso lines numbered as described in FIG. 12A. The treatment area 2215 has a treatment value at the 4.0 iso line to a depth ranging from 5.0 to 7.25. While the range is a bit larger as shown in FIG. 22B than 22A, the overall distribution of the treatment appears more even across the treatment area 2265. Accordingly, the distribution of the treatment is improved when a recess is used as shown in FIG. 22B over a conductive spacer without recesses as shown in FIG. 22A.

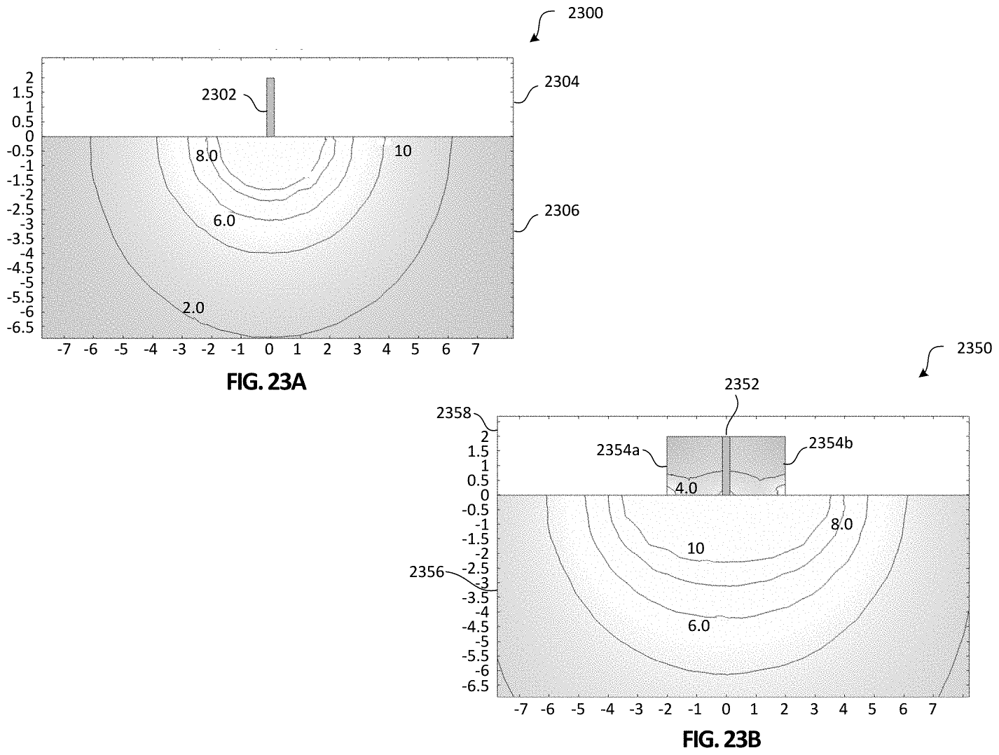

FIGS. 23A and 23B depict models of treatment areas using monopolar electrode assemblies.

FIG. 23A illustrates a model 2300 of a treatment area 2306 for an electrode assembly having a monopolar electrode 2302 with no conductive spacer. A voltage pulse is applied that may have, for example, 1 kV to 15 kV peak voltage. The approximate treatment values at various depths are shown by the iso lines numbered as described in FIG. 12A. The treatment area 2306 has a treatment value at the 4.0 iso line to a depth of 4.0, but the shape is quite rounded, such that the deepest area is only at one point in the center of the treatment area.

FIG. 23B illustrates a model 2350 of a treatment area 2356 for an electrode assembly having a monopolar electrode 2352 with a conductive spacer 2354 disposed around the monopolar electrode. In some embodiments, the conductive spacer 2354 may surround the monopolar electrode 2352 in a ring shape. A voltage pulse is applied that may have, for example, up to 20 kV peak voltage. The approximate treatment values at various depths are shown by the iso lines numbered as described in FIG. 12A. The treatment area 2356 has a treatment value at the 4.0 iso line to a depth of 4.25. Notice that with the conductive spacer, the distribution of the treatment in the treatment area 2356 is substantially less rounded and more evenly distributed than the treatment depicted in FIG. 23A.

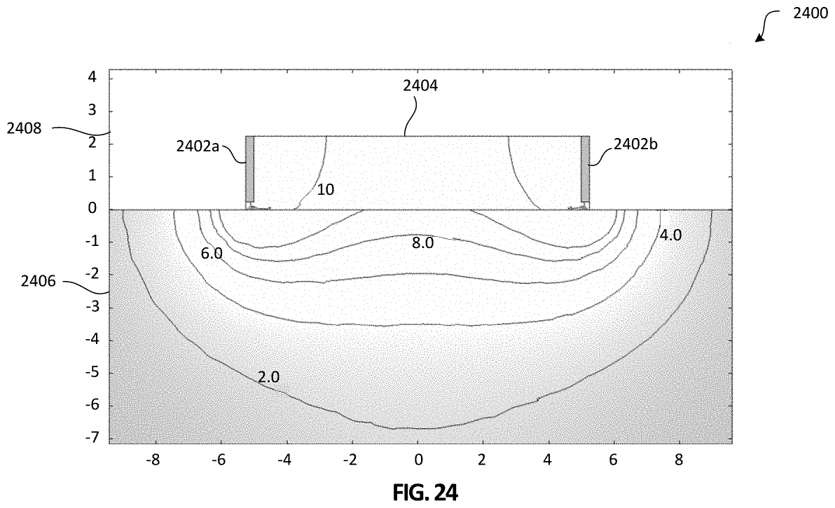

FIG. 24 illustrates a model 2400 of a treatment area 2406 for an electrode assembly having two electrodes 2402a and 2402b and a conductive spacer 2404. Conductive spacer 2404 extends between the electrodes 2402 and is further disposed on the top edge of the electrodes 2402, such that the conductive spacer touches the treatment area 2406 directly and the electrodes 2402 do not directly contact the treatment area 2406. The electrodes 2402 provide the treatment to the treatment area 2406 indirectly through the conductive spacer 2404. A voltage pulse is applied that may have, for example, 1 kV to 15 kV peak voltage. The approximate treatment values at various depths are shown by the iso lines numbered as described in FIG. 12A. The treatment area 2406 has a treatment value at the 4.0 iso line to a depth of 3.5, which is relatively consistent across the treatment area 2406.

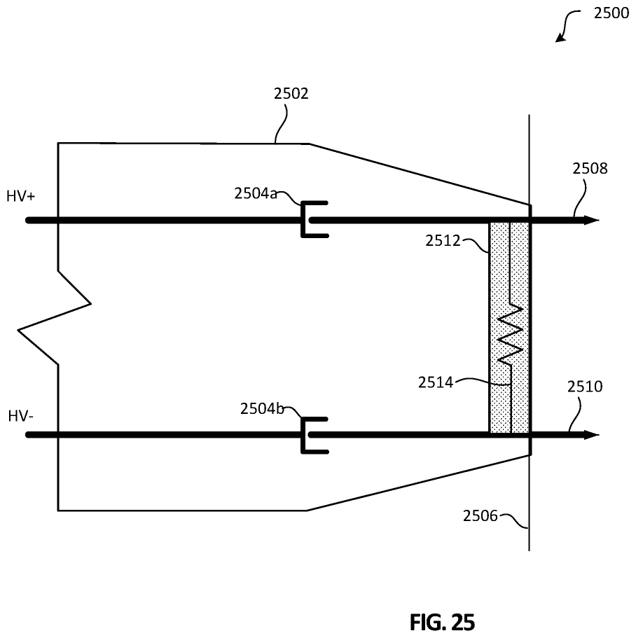

FIG. 25 demonstrates by example another inventive concept of the present disclosure. To optimize the pulse shape, a parallel resistor may be used in some electrode assemblies (e.g., treatment tips) to decrease the treatment tip impedance to better match the system impedance. To accomplish this, in previously existing systems which use electrode assemblies that are connected to a handle of the treatment applicator, one or more resistors is typically placed in the handle and is positioned to be in parallel with the electrode assembly/treatment tip once it is connected to the handle. Coupling of the electrode assembly to the handle portion of the treatment applicator actuates the resistor in the handle portion. Such configuration adds complexity to the handle portion of the treatment applicator and, in addition, in some implementations may limit the value of the parallel resistor in the handle portion. Using a novel configuration of the present disclosure, which is shown by example in FIG. 25, resolves the above issues, may improve/simplify the parts and manufacturing, and allows various electrode assemblies to be custom tuned.

FIG. 25 illustrates a cross-sectional view of an example of the treatment applicator 2500 with the electrode assembly 2502 according to the present disclosure. Treatment applicator assembly 2500 may include a handle portion 2505 that may be releasably connected to different treatment tips (different electrode assemblies). A positive high voltage input HV+ of the handle portion 2505 may couple via connector 2504a to an electrode 2502a of the treatment tip and a negative high voltage input HV− of the handle portion may couple via connector 2504b to an electrode 2502b of the treatment tip. In this example electrodes 2502a and 2502b are shown as the needle electrodes and may penetrate tissue 2506, however, as explained above, various configurations of the electrode assembly (penetrating and non-penetrating) may be used in different examples. Instead of having one or more separate resistors in the handle portion, a conductive spacer 2512 positioned in the treatment tip/electrode assembly may be configured to play a double role and function as both: the conductive spacer and also as a resistor 2512′. The conductive spacer/resistor 2512/2512′ may be conductively coupled between the electrode 2502a and the electrode 2502b. Electrodes 2502a and 2502b may be, for example, similar to the needle electrodes 425 as described with respect to FIG. 4B. Conductive spacer/resistor may be a semi-conductive material, such as conductive plastic, making the conductive spacer 2512 also function as a parallel resistor.

It may be desirable to have resistance, for example, between 10 and 800 Ohms (e.g., between 10 and 500 Ohms, between 100 and 500 Ohms, between 100 and 400 Ohms, between 100 and 800 Ohms, between 150 and 350 Ohms, between 150 and 800 Ohms, etc.). The resistor value (as parallel resistor), and conductive spacer value (as conductive spacer to improve treatment depth) can be tuned by choosing the conductivity of the material and/or by changing the geometry of the conductive spacer/resistor. As disclosed above, the shape of the conductive spacer can change the shape of the treatment zone. The shape of the conductive spacer may also be used to change the value of the parallel resistance. For example, smaller cross-sectional area would result in higher resistance, larger cross-sectional area would result in lower resistance. As an example, the tip in FIG. 8A would have a lower parallel resistance value than the tip in FIG. 8B. Also, smaller distance between the HV+ and HV− gives lower resistance. Therefore, for example, with the exact same cross-sectional area, a smaller (e.g., 2.5 mm) treatment tip would have a lower parallel resistance value than a larger (e.g., 5 mm) treatment tip. In light of the above, depending on the requirements and implementations, a cross sectional area may be adjusted or the conductivity of the spacer material may be changed to optimize the dual function of the conductive spacer to also function as an effective resistor. The spacer/resistor as described in reference to FIG. 25 may be implemented in any of the other examples and embodiments of the electrode assemblies, treatment tips/treatment applicators and electrical therapy devices of the present disclosure.

In any of the methods of the present disclosure, including those described in reference to FIGS. 10A-10B and 11, the method may comprise using the conductive spacer also as a resistor by selecting the electrode assembly and/or the conductive spacer to optimize both a desired resistance value and a treatment depth. Therefore, the conductivity of the material and/or geometry of the conductive spacer may be selected to improve a treatment depth, or a value of the resistance, or both. Any such method may comprise tuning a value of the conductive spacer as a resistor by changing the conductivity of the material and/or by changing the geometry of the resistor. Also, the method may comprise tuning a value of a conductive spacer to improve treatment depth by changing the conductivity of the material and/or by changing the geometry of the conductive spacer. The method may comprise using a shape of the conductive spacer to change a value of the parallel resistance. In various examples, the method may include using the conductive spacer as a resistor to match an impedance of an electrode assembly to an impedance of a pulse generator that generates and applies electrical energy via the exchangeable electrode assembly. In some examples, the method may include using the conductive spacer to reduce an impedance of the electrode assembly. In various examples, the methods of the present disclosure may comprise selecting a shape of the conductive spacer to change a shape of the treatment zone or area.

Any of the methods (including user interfaces) described herein may be implemented as software, hardware or firmware, and may be described as a non-transitory computer-readable storage medium storing a set of instructions capable of being executed by a controller that includes one or more processor (e.g., computer, tablet, smartphone, etc.), that when executed by the controller/processor causes the controller/processor to control performance or perform any of the steps, including but not limited to: displaying, communicating with the user, analyzing, modifying parameters (including timing, frequency, intensity, etc.), determining, alerting, or the like.

Although various illustrative embodiments are described above, any of a number of changes may be made to various embodiments without departing from the scope of the disclosure. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

It is understood that the various embodiments described herein are by way of example only and are not intended to limit the scope of the invention. For example, many of the materials and structures described herein may be substituted with other materials and structures without deviating from the spirit of the invention as would be apparent to those skilled in the art upon review of the disclosure. Thus, although specific embodiments (examples) have been illustrated and described herein, any arrangement calculated to achieve the same purpose may be substituted for the specific examples shown. This disclosure is intended to cover any and all adaptations or variations of various examples. Combinations of the above examples or some features of the described examples, and other examples not specifically described herein, will be apparent to those of skill in the art upon reviewing the above description. The present invention as claimed may therefore include variations from the particular examples and embodiments described herein. It is understood that various theories as to why the invention works are not intended to be limiting. Various embodiments of the inventive subject matter may be referred to herein individually or collectively by the term “invention” merely for convenience and without intending to voluntarily limit the scope of this application to any single invention or inventive concept, if more than one is, in fact, disclosed.

All measurements, dimensions, and materials provided herein within the specification or within the figures are by way of example only. As used herein in the specification and claims, including as used in the examples and unless otherwise expressly specified, all numbers may be read as if prefaced by the word “about” or “approximately,” even if the term does not expressly appear.

A recitation of “a,” “an,” or “the” is intended to mean “one or more” unless specifically indicated to the contrary. Reference to a “first” component does not necessarily require that a second component be provided. Moreover, reference to a “first” or a “second” component does not limit the referenced component to a particular location unless expressly stated. When a feature or element is herein referred to as being “on” another feature or element, it can be directly on the other feature or element or intervening features and/or elements may also be present. It will also be understood that, when a feature or element is referred to as being “connected”, “attached” or “coupled” to another feature or element, it can be directly connected, attached or coupled to the other feature or element or intervening features or elements may be present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

1. An electrode assembly for delivery of electrical therapy, the electrode assembly comprising:
at least two electrodes, each electrode comprising:
a conductive treatment surface configured to apply electrical treatment to a treatment area, and
a conductive non-treatment surface configured to contact a conductive spacer; and
the conductive spacer disposed between the at least two electrodes, wherein the conductive spacer is configured to electrically contact conductive non-treatment surfaces of the at least two electrodes and a surface of the treatment area between the at least two electrodes.

2. The electrode assembly of claim 1, wherein the conductive spacer comprises one of a hydrogel, a conductive adhesive, a conductive gel, a conductive silicone, a urethane rubber, thermoplastic, thermoset resin, or any combination thereof.

3. The electrode assembly of claim 1, wherein a conductivity of the conductive spacer is substantially equal to ten times (10×) a conductivity of a tissue of the treatment area.

4. The electrode assembly of claim 1, wherein a conductivity of the conductive spacer is greater than or substantially equal to a conductivity of a tissue of the treatment area and less than or equal to one hundred times (100×) the conductivity of the tissue of the treatment area.

5. The electrode assembly of claim 1, wherein a height of the conductive spacer is based on a distance between the at least two electrodes.

6. The electrode assembly of claim 1, wherein a height of the conductive spacer is greater than or equal to twenty percent (20%) of a distance between the at least two electrodes and less than or equal to fifty percent (50%) of the distance between the at least two electrodes.

7. The electrode assembly of claim 1, wherein the conductive treatment surface of the at least two electrodes electrically and/or directly contacts the treatment area.

8. The electrode assembly of claim 1, wherein the at least two electrodes comprise bipolar electrodes or monopolar electrodes.

9. The electrode assembly of claim 1, wherein at least one of the at least two electrodes comprises a surface electrode.

10. The electrode assembly of claim 1, wherein at least one of the at least two electrodes comprises a penetrating electrode.

11. The electrode assembly of claim 1, wherein a length of the conductive spacer is substantially equal to a length of one or more of the at least two electrodes.

12. The electrode assembly of claim 1, wherein the at least two electrodes comprise two rows of needle electrodes, and wherein a length of the conductive spacer is substantially equal to a length of one or both of the two rows.

13. The electrode assembly of claim 1, wherein the conductive spacer comprises at least two conductive zones, each of the conductive zones having a different conductivity.

14. The electrode assembly of claim 1, wherein the conductive spacer comprises at least one recess.

15. The electrode assembly of claim 1, wherein the conductive spacer comprises a semi-conductive material and is configured to act as a parallel resistor.

16. An electrical therapy device comprising:
a treatment applicator configured to be coupled to one of a plurality of exchangeable electrode assemblies; and
the plurality of exchangeable electrode assemblies, each of the plurality of exchangeable electrode assemblies comprising:
at least two electrodes, each electrode comprising:
a conductive treatment surface configured to apply electrical treatment to a treatment area of a subject, and
a conductive non-treatment surface configured to contact a conductive spacer; and
the conductive spacer disposed between the at least two electrodes, wherein the conductive spacer is configured to electrically contact conductive non-treatment surfaces of the at least two electrodes and a surface of the treatment area of the subject between the at least two electrodes.

17. A method of administering electrical therapy, the method comprising:
positioning an electrode assembly comprising at least two electrodes and a conductive spacer disposed between the at least two electrodes on a treatment area such that:
a conductive treatment surface of each of the at least two electrodes electrically contacts the treatment area, and
the conductive spacer contacts a surface of the treatment area between the at least two electrodes; and
applying a voltage to the treatment area via the electrode assembly.

18. The method of claim 17, the method comprising selecting the electrode assembly based on the conductive spacer having a conductivity that is substantially equal to ten times (10×) a conductivity of a tissue of the treatment area.

19. The method of claim 17, the method comprising selecting the electrode assembly based at least in part on a size of the electrode assembly and/or a size of the treatment area and/or selecting the voltage to be applied to the treatment area via the electrode assembly based at least in part on a conductivity of the conductive spacer.

20. The method of claim 17, further comprising:
selecting the electrode assembly from a plurality of electrode assemblies based at least in part on a conductivity of a material and/or a geometry of the conductive spacer.
